(19) United States
(12) Patent Application Publication
(10) Pub. No.: US 2006/0241149 A1
(43) Pub. Date: Oct. 26, 2006

(54) Chemical compounds

(76) Inventors: Jerry Leroy Adams, Collegeville, PA (US); Deborah L. Bryan, King of Prussia, PA (US); Jiri Kasparec, King of Prussia, PA (US); Francis David King, King of Prussia, PA (US); Andrew Kenneth Takle, King of Prussia, PA (US); David Matthew Wilson, King of Prussia, PA (US); Steven Neal Goodman, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/533,832

(22) PCT Filed: Nov. 6, 2003
(86) PCT No.: PCT/US03/35588

§ 371 Date: May 3, 2005

Related U.S. Application Data
(60) Provisional application No. 60/424,223, filed on Nov. 6, 2002.

(51) Int. Cl. A61K 31/4439; C07D 403/02

(52) U.S. Cl. 514/337; 546/277.4

(57) ABSTRACT

Pyridinyl-benzoheterocycyl kinase inhibitor compounds are disclosed. Also disclosed are methods of making such compounds as well as methods of using the same in the treatment of diseases.

### BACKGROUND OF THE INVENTION

The present invention relates to pyridinyl-benzoheterocycyl derivatives, compositions and medicaments containing the same, as well as processes for the preparation and use of such compounds, compositions and medicaments. Such pyridinyl-benzoheterocycyl derivatives are useful in the treatment of diseases associated with inappropriate angiogenesis.

The process of angiogenesis is the development of new blood vessels, generally capillaries, from preexisting vasculature. Angiogenesis is defined as involving: (i) activation of endothelial cells; (ii) increased vascular permeability; (iii) subsequent dissolution of the basement membrane and extravisation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vii) deposition of basement membrane and recruitment of perivascular cells to newly formed vessels. Normal angiogenesis is activated during tissue growth, from embryonic development through maturity, and then enters a period of relative quiescence during adulthood. Normal angiogenesis is also activated during wound healing, and at certain stages of the female reproductive cycle. Inappropriate angiogenesis has been associated with several disease states including various retinopathies; ischemic disease; atherosclerosis; chronic inflammatory disorders; and cancer. The role of angiogenesis in disease states is discussed, for instance, in Fan et al., Trends in Pharmacol. Sci. 16: 54-66; Shawver et al., DDT Vol. 2, No. 2 Feb. 1997; Folkmann, 1995, Nature Medicine 1: 27-31; Colville-Nash and Scott, Ann. Rheum. Dis., 51, 919,1992; Brooks et al., Cell, 79, 1157, 1994; Kahlon et al., Can. J. Cardiol. 8, 60, 1992; Folknan, Cancer Biol, 3, 65, 1992; Denekamp, Br. J. Rad. 66, 181, 1993; Fidler and Ellis, Cell, 79, 185, 1994; O'Reilly et al., Cell, 79, 315, 1994; Ingber et al., Nature, 348, 555, 1990; Friedlander et al., Science, 270, 1500, 1995; Peacock et al., J. Exp. Med. 175, 1135, 1992; Peacock et al., Cell. Immun. 160, 178, 1995; and Taraboletti et al., J. Natl. Cancer Inst. 87, 293, 1995.

In cancer the growth of solid tumors has been shown to be angiogenesis dependent. (See Folkmann, J., J. Nat'l. Cancer Inst., 1990, 82, 4-6.) Consequently, the targeting of pro-angiogenic pathways is a strategy being widely pursued in order to provide new therapeutics in these areas of great, unmet medical need. The role of tyrosine kinases involved in angiogenesis and in the vascularization of solid tumors has drawn interest. Until recently most interest in this area has focused on growth factors such as vascular endothelial growth factor (VEGF) and its receptors termed vascular endothelial growth factor receptor(s) (VEGFR). VEGF, a polypeptide, is nitogenic for endothelial cells in vitro and stimulates angiogenic responses in vivo. VEGF has also been linked to inappropriate angiogenesis (Pinedo, H. M. et al. The Oncologist, Vol.5, No. 90001, 1-2, April 2000). VEGFR(s) are protein tyrosine kinases (PTKs). PTKs catalyze the phosphorylation of specific tyrosyl residues in proteins involved in the regulation of cell growth and differentiation. (A. F. Wilks, Progress in Growth Factor Research, 1990, 2, 97-111; S. A. Courtneidge, Dev. Supp. 1, 1993,57-64; J. A. Cooper, Sernin. Cell Biol., 1994, 5(6),377-387; R. F. Paulson, Semin. Immunol., 1995, 7(4),267-277; A. C. Chan, Curr. Opin. Immunol., 1996, 8(3), 394-401).

Three PTK receptors for VEGF have been identified: VEGFR-1 (Flt-1); VEGFR-2 (Flk-1 or KDR) and VEGFR-3 (Flt-4): These receptors are involved in angiogenesis and participate in signal transduction (Mustonen, T. et al. J. Cell Biol. 1995, 129: 895-898). Of particular interest is VEGFR-2, which is a transmembrane receptor PTK expressed primarily in endothelial cells. Activation of VEGFR-2 by VEGF is a critical step in the signal transduction pathway that initiates tumor angiogenesis. VEGF expression may be constitutive to tumor cells and can also be upregulated in response to certain stimuli. One such stimuli is hypoxia, where VEGF expression is upregulated in both tumor and associated host tissues. The VEGF ligand activates VEGFR-2 by binding with its extracellular VEGF binding site. This leads to receptor dimerization of VEGFRs and autophosphorylation of tyrosine residues at the intracellular kinase domain of VEGFR-2. The kinase domain operates to transfer a phosphate from ATP to the tyrosine residues, thus providing binding sites for signaling proteins downstream of VEGFR-2 leading ultimately to initiation of angiogenesis (McMahon, G., The Oncologist, Vol.5, No. 90001, 3-10, April 2000).

Angiopoietin 1 (Ang1), a ligand for the endothelium-specific receptor tyrosine kinase TIE-2 is a novel angiogenic factor (Davis et al., Cell, 1996, 87: 1161-1169; Partanen et al., Mol. Cell Biol., 12: 1698-1707 (1992); U.S. Pat Nos. 5,521,073; 5,879,672; 5,877,020; and 6,030,831). The acronym TIE represents “tyrosine kinase containing Ig and EGF homology domains”. TIE is used to identify a class of receptor tyrosine kinases, which are exclusively expressed in vascular endothelial cells and early hemopoietic cells. Typically, TIE receptor kinases are characterized by the presence of an EGF-like domain and an immunoglobulin (IG) like domain, which consists of extracellular folding units, stabilized by intra-chain disulfide bonds (Partanen et al., Curr. Topics Microbiol. Immunol., 1999, 237: 159-172). Unlike VEGF, which functions during the early stages of vascular development, Ang1 and its receptor TE-2 function in the later stages of vascular development, i.e., during vascular remodeling (remodeling refers to formation of a vascular lumen) and maturation (Yancopoulos et al., Cell, 1998, 93: 661-664; Peters, K. G., Circ. Res., 1998, 83(3): 342-3; Suri et al., Cell 87, 1996: 1171-1180).

Consequently, inhibition of TIE-2 would be expected to serve to disrupt remodeling and maturation of new vasculature initiated by angiogenesis thereby disrupting the angiogenic process. Furthermore, inhibition at the kinase domain binding site of VEGFR-2 would block phosphorylation of tyrosine residues and serve to disrupt initiation of angiogenesis. Presumably then, inhibition of TIE-2 and/or VEGFR-2 should prevent tumor angiogenesis and serve to retard or eradicate tumor growth. Accordingly, a treatment for cancer or other disorder associated with inappropriate angiogenesis could be provided. Inhibitors of Raf kinases have been suggested for use in disruption of tumor cell growth and hence in the treatment of cancers, e.g., melanoma, histiocytic lymphoma, lung adenocarcinoma, colorectal, ovarian, and small cell lung cancer and pancreatic and breast carcinoma; (Helen Davies et al., Nature, 2002, 417: 949. Activated cell surface receptors activate ras/rap proteins at the inner aspect of the plasma-membrane which in turn recruit and activate Raf proteins. Activated Raf proteins phosphorylate and activate the intracellular protein kinases MEK1 and MEK2. In turn, activated MEKs catalyse phosphorylation and activation of p42/p44 mitogen-activated protein kinase (MAPK). A variety of cytoplasmic and nuclear substrates of activated MAPK are known which directly or indirectly contribute to the cellular response to environmental change. Three distinct genes have been identified in mammals that encode Raf proteins; A-Raf, B-Raf and C-Raf (also known as Raf-1) and isoformic variants that result from differential splicing of mRNA are known. Presumably then, inhibition of Raf kinase should serve to retard or eradicate tumor growth. Accordingly, a treatment for cancer could be provided.

The pyridinyl-benzoheterocycyl compounds are inhibitors of one or more of TIE-2 kinase activity, VEGFR-2 kinase activity, VEGFR-3 kinase activity or Raf kinase activity. Such pyridinyl-benzoheterocycyl derivatives are useful in the treatment of disorders, mediated by at least one of inappropriate TIE-2 kinase, VEGFR-2 kinase, VEGFR-3 activity or Raf kinase activity (which may include cancer and/or diseases afflicting mammals which is characterized by cellular proliferation in the area of disorders associated with neo-vascularization and/or vascular permeability), and/or disorders characterized by inappropriate angiogenesis; and/or for treating cancer and/or a disease afflicting afflicting mammals which is characterized by cellular proliferation in the area of disorders associated with neo-vascularization and/or vascular permeability.

### SUMMARY OF THE INVENTION

This invention is directed to a compound of Formula I: 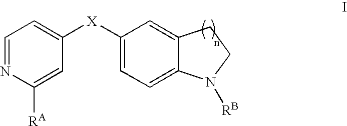
wherein:

n is an integer of 1,2, or 3;

R^{A} is —CONHR¹, —NHR¹, —NHCOR¹, —NHCONHR¹, —NHCO₂R¹, —NHSO₂R¹ or —NHSO₂NHR¹;

wherein R¹ is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ allyl- or heteroaryl-C₁-C₄ alkyl- group,

where said optionally substituted R¹ group is optionally substituted with one or more substituents independently selected from halogen, —R^{1a}, —OR^{1a}, —SR^{1a}, —SO₂R^{1c} —NR^{1a}R^{1b}, cyano, nitro, —COR^{1c}, —CO₂R^{1a}, —NR^{1b}COR^{1a}, —CONR^{1a}R^{1b}, —NR^{1b}SO₂R^{1c}, and —SO₂NR^{1a}R^{1b},

where R^{1a} is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,

R^{1b} is hydrogen or unsubstituted C₁-C₄ alkyl, and

R^{1c} is an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂C₄ alkynyl-, C₃-C₇ cycloalkyl-C₂-C₄ alkynyl-, heteroaryl-C₂-C₄ alkynyl-, or heterocycyl-C₂-C₄ alkynyl- group,

where each optionally substituted R^{1a} group and R^{1c} group is independently optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,

or R^{1a} and R^{1b}, together with the nitrogen atom to which they are attached, form an optionally substituted heterocycyl or heteroaryl ring which optionally contains one or more additional heteroatom moieties selected from O, S, SO, SO₂, N and N→O, wherein said optionally substituted heterocycyl or heteroaryl ring is optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄alkyl,

X is NR², O, S, SO or SO₂,

wherein R² is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl- or heteroaryl-C₁-C₄ alkyl- group,

where said optionally substituted R² group is optionally substituted with one or more substituents independently selected from halogen, —R^{2a}, OR^{2a}, —SR^{2a}, —SO₂R^{2c} —NR^{2a}R^{2b}, cyano, nitro, —COR^{2c}, —CO₂R^{2a}, —NR^{2b}COR^{2a}, —CONR^{2a}R^{2b}, —NR^{2b}SO₂R^{2c}, and —SO₂NR^{2a}R^{2b},

where R^{2a} is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,

R^{2b} is hydrogen or unsubstituted C₁-C₄ alkyl, and

R^{2c} is an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,

where each optionally substituted R^{2a} group and R^{2c} group is independently optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,

or R^{2a} and R^{2b}, together with the nitrogen atom to which they are attached, form an optionally substituted heterocycyl or heteroaryl ring which optionally contains one or more additional heteroatom moieties selected from O, S, SO, SO₂, N and N→O, wherein said optionally substituted heterocycyl or heteroaryl ring is optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,

R^{B} is —CONHR³, —SO₂R³; —CO₂R³, —COC(R⁴R⁵)R³,

wherein R³ is hydrogen or an optionally substituted C₁-C₆ alkyl, C₂-C₆ alkenyl, C₂-C₆ alkynyl, aryl, C₃₋C₇ cycloalkyl, heteroaryl, heterocyclyl, aryl-C₁-C₄ alkyl- or heteroaryl-C₁-C₄ alkyl- group,

where said optionally substituted R³ group is optionally substituted with one or more substituents independently selected from halogen, —R^{3a}, —OR^{3a}, —SR^{3a}, —SO₂R^{3c} —NR^{3a}R^{3b}, cyano, nitro, —COR^{3c}, —CO₂R^{3a}, —NR^{3b}COR^{3a}, —CONR^{3a}R^{3b}, —NR^{3b}SO₂R^{3c}, and —SO₂NR^{3a}R^{3b},

where R3a is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,

R^{3b} is hydrogen or unsubstituted C₁-C₄ alkyl, and

R^{3c} is an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkenyl-, C₃-C₇ cycloalkyl-C₂-C₄ alkynyl-, heteroaryl-C₂-C₄ alkynyl-, or heterocycyl-C₂-C₄ alkynyl- group,

where each optionally substituted R^{3a} group and R^{3c} group is independently optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,

or R^{3a} and R^{3b}, together with the nitrogen atom to which they are attached, form an optionally substituted heterocycyl or heteroaryl ring which optionally contains one or more additional heteroatom moieties selected from O, S, SO, SO₂, N and N→O, wherein said optionally substituted heterocycyl or heteroaryl ring is optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ allyl is unsubstituted C₁-C₄ alkyl, and

R⁴ and R⁵ are independently selected from hydrogen and unsubstituted C₁-C₄ alkyl,

or R⁴ and R⁵, taken together with the carbon atom to which they are attached, represent an optionally substituted 3-6membered saturated carbocyclic ring, where said optionally substituted 3-6membered ring is substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,

or a salt, solvate, or physiologically functional derivative thereof.

This invention is also directed to a pharmaceutical composition comprising a therapeutically effective amount of a compound of Formula I, or a salt, solvate, or a physiologically functional derivative thereof and one or more of pharmaceutically acceptable carriers, diluents and excipients. In another embodiment, this invention is directed to a method of treating a disorder in a mammal, said disorder being mediated by at least one of inappropriate TIE-2, VEGFR-2 VEGFR-3 and Raf kinase activity, comprising: administering to said mammal a therapeutically effective amount of a compound of Formula I or a salt, solvate or a physiologically functional derivative thereof. In yet another embodiment, this invention is directed to a compound of Formula I, or a salt, solvate, or a physiologically functional derivative thereof for use in therapy. In another embodiment, this invention is directed to the use of a compound of Formula I, or a salt, solvate, or a physiologically functional derivative thereof in the preparation of a medicament for use in the treatment of a disorder mediated by at least one of inappropriate TIE-2, VEGFR-2, VEGFR-3 or Raf kinase activity. In a further aspect of this invention, there is provided a method of treating a disorder in a mammal, said disorder being mediated by at least one of inappropriate TIE-2, VEGFR-2, VEGFR-3 and Raf kinase activity, comprising: administering to said mammal therapeutically effective amounts of (i) a compound of Formula I, or a salt, solvate or physiologically functional derivative thereof and (ii) an agent to inhibit growth factor receptor function. This invention is also directed to a method of treating a disorder in a mammal, said disorder being characterized by inappropriate angiogenesis, comprising: administering to said mammal a therapeutically effective amount of a compound of Formula I, or a salt, solvate or physiologically functional derivative thereof.

### DETAILED DESCRIPTION OF THE INVENTION

All documents cited or referred to herein, including issued patents, published and unpublished patent applications, and other publications are hereby incorporated herein by reference as though fully set forth.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “alkyl” refers to a straight or branched chain saturated hydrocarbon radical having from one to twelve carbon atoms, unless otherwise specified, optionally substituted with one or more substituents as defined herein. Examples of “alkyl” as used herein include, but are not limited to, methyl, ethyl, n-propyl, isopropyl, n-butyl, isobutyl, t-butyl, n-pentyl, isopentyl, and the like. The term “C₁-C₆ alkyl” refers to an alkyl group as defined above containing at least 1, and at most 6, carbon atoms. Examples of branched or straight chained “C₁-C₆ alkyl” groups useful in the present invention include, but are not limited to, methyl, ethyl, n-propyl, isopropyl, isobutyl, n-butyl, t-butyl, n-pentyl, and isopentyl.

As used herein, the term “alkenyl” refers to a straight or branched chain hydrocarbon radical having from two to ten carbons, unless otherwise specified, and at least one carbon-carbon double bond, optionally substituted with one or more substituents as defined herein. Examples of “alkenyl” as used herein include ethenyl, propenyl, 1-butenyl, 2-butenyl, and isobutenyl. The term “C₂-C₆ alkenyl” refers to an alkenyl group as defined above containing at least 2, and at most 6, carbon atoms. Examples of “C₂-C₆ alkenyl” groups useful in the present invention include, but are not limited to, ethenyl, propenyl, 1-butenyl, 2-butenyl, and isobutenyl.

“Alkynyl” refers to a straight or branched chain hydrocarbon radical having from two to ten carbons, unless otherwise specified, and at least one carbon-carbon triple bond, optionally substituted with one or more substituents as defined herein. Examples of “alkynyl” as used herein, include but are not limited to acetylenyl, 1-propynyl, 1-butynyl, 2-butynyl, 1-pentynyl, and 1-hexynyl.

The term “halogen” refers to fluorine (F), chlorine (Cl), bromine (Br), or iodine (I) and the term “halo” refers to the halogen radicals fluoro, chloro, bromo, and iodo.

As used herein, the term “C₁-C₆ haloalkyl” refers to an alkyl group as defined above containing at least 1, and at most 6, carbon atoms substituted with at least one halo group, halo being as defined herein. Examples of branched or straight chained “C]-C₆ haloalkyl” groups useful in the present invention include, but are not limited to, methyl, ethyl, propyl, isopropyl, isobutyl and n-butyl substituted independently with one or more halos, e.g., fluoro, chloro, bromo and iodo, e.g., trifluoromethyl.

As used herein, the term “C₃-C₇ cycloalkyl” refers to a non-aromatic cyclic hydrocarbon radical having from three to seven carbon atoms which may be saturated or partially unsaturated and which is optionally substituted with one or more substituents as defined herein. Exemplary “C₃-C₇ cycloalkyl” groups include, but are not limited to, cyclopropyl, cyclobutyl, cyclopentyl, cyclopentenyl, cyclopentadienyl, cyclohexyl and cycloheptyl.

As used herein, the term “aryl” refers to an optionally substituted benzene ring or to an optionally substituted benzene ring fused to one or more optionally substituted benzene rings to form a ring system, which rings are optionally substituted with one or more substituents as defined herein. Such a ring or ring system may be optionally fused to one or more optionally substituted aryl rings (including benzene rings) or cycloalkyl rings. Examples of “aryl” groups include, but are not limited to, phenyl, naphthyl, tetrahydronaphthyl, indanyl and indenyl, as well as substituted derivatives thereof.

As used herein, the term “heterocyclic” or the term “heterocyclyl” refers to a three to twelve-membered ring containing one or more heteroatornic ring moieties selected from S, SO, SO₂, O , N, or N-oxide, optionally substituted with one or more substituents as defined herein. Such a ring can be saturated or have one or more degrees of saturation. Such a ring may be optionally fused to one or more other optionally substituted, “heterocyclic” ring(s) or cycloalkyl ring(s). Examples of “heterocyclic” moieties include, but are not limited to, tetrahydrofuranyl, pyranyl, 1,4-dioxyl, 1,3-dioxyl, piperidinyl, pyrrolidinyl, morpholinyl, tetrahydrothiopyranyl, tetrahydrotbienyl, and the like.

As used herein, the term “heteroaryl” refers to an optionally substituted monocyclic five to seven membered aromatic ring containing one or more heteroatomic ring moieties selected from S, SO, SO₂, O, N, or N-oxide, or to such an aromatic ring fused to one or more, optionally substituted, heteroaryl rings, aryl rings (including benzene rings), heterocyclic rings, or cycloalkyl rings (e.g., a bicyclic or tricyclic ring system), which rings are optionally substituted with one or more substituents as defined herein. Examples of “heteroaryl” groups used herein include, but are not limited to, furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, tetrahydroisoquinolinyl, benzofuranyl, dihydrobenzofuranyl, benzothiophenyl, dihydrobenzothienyl, indolyl, indazolyl, and substituted versions thereof.

When the term “alkyl” (or alkenyl or alkynyl) is used in combination with other substituent groups, such as “haloalkyl,” “aryl-C₁-C₄ alkyl-,” “aryl-C₂-C₄ alkenyl-,” or “heteroaryl-C₁-C₄ alkyl-”, the term “alkyl” (or alkenyl or alkynyl) is intended to encompass a divalent straight or branched-chain hydrocarbon radical. For example, “cycloalkylalkyl” is intended to mean the radical -alkyl-cycloalkyl, wherein the alkyl moiety thereof is a divalent straight or branched-chain saturated hydrocarbon radical and the cycloalkyl moiety thereof is as defined herein, and is represented by the bonding arrangement present in the groups —CH₂-cyclopropyl, —CH₂-Cyclohexyl, or —CH₂(CH₃)CHCH₂-cyclopentenyl. Examples of “aryl-C₁-C₄ alkyl-” include, but are not limited to, benzyl and phenylpropyl. Examples of “heteroaryl-C₁-C₄ alkyl-” include, but are not limited to, 2-pyridylmethyl, 3-isoxazolylmethyl, 3-(1-methyl-5-t-butyl-pyrazoyl)methyl, 3-isoxazolylmethyl, and 2-imidazolyl ethyl. Examples of “heterocycyl-C₁-C₄ alkyl-” include, but are not limited to, 1-methyl-piperidinylpropyl, morpholinoethyl, morpholinopropyl, pyrrolidinonyl-butyl, pyrrolidinyl-butyl, and pyrrolidinyl-pentyl. The term “aryl-C₂-C₄ alkenyl-” is intended to mean the radical -alkenyl-aryl, wherein the alkenyl moiety thereof is a divalent straight or branched-chain hydrocarbon radical containing two to four carbon atoms and at least one carbon-carbon double bond and the aryl moiety thereof is as defined herein, and is represented by the bonding arrangement present in a styryl group, e.g., —CH═CH-phenyl.

It will be understood that when a group or moiety is “optionally substituted,” the group or moiety may be unsubstituted or may be substituted by one or more of the substituents defined herein, where each substituent is selected independently.

As used herein, the term “physiologically functional derivative” refers to any pharmaceutically acceptable derivative of a compound of the present invention, for example, an ester or an amide, which upon administration to a manual is capable of providing (directly or indirectly) a compound of the present invention or an active metabolite thereof. Such derivatives are clear:to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of Formula I or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by Formula I above as well as any wholly or partially equilibrated mixtures thereof. The present invention also covers the individual isomers of the compounds represented by the formulas above as mixtures with isomers thereof in which one or more chiral centers are inverted. Also, it is understood that all tautomers and mixtures of tautomers of the compounds of Formula I are included within the scope of the compounds of Formula I.

In one particular embodiment of the present invention are compounds having Formula I wherein n is 1 or 2. In another particular embodiment of the invention are compounds of Formula I wherein n is 1. In another embodiment of the invention are compounds of Formula I wherein X is O or NR². In yet another embodiment of the invention are compounds of Formula I wherein X is O.

Another embodiment of the compounds of Formula I are compounds of Formula II:

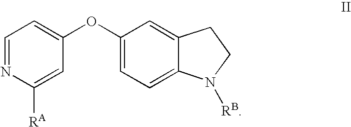

More specific embodiments of the compounds of Formula I are compounds of Formula III

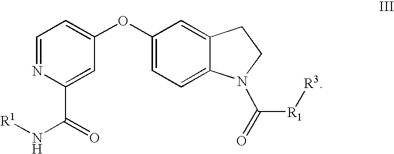

In one embodiment of the invention, R^{A} is —CONHR¹, —NHCOR¹, or —NHSO₂R¹, where R¹ is C₁-C₆ alkyl, aryl, heteroaryl, heterocycyl, aryl-C₁-C₄ alkyl-, heteroaryl-C₁-C₄ alkyl-, or heterocycyl-C₁-C₄ alkyl-, wherein said C₁-C₆ alkyl is optionally substituted with one ore more substituents independently selected from —NH₂, —N(C₁-C₄ alkyl)(C₁-C₄ alkyl), and —NH(C₁-C₄ alkyl), or said aryl, heteroaryl or heterocycyl or the aryl, heteroaryl or heterocycyl moiety of said aryl-C₁-C₄ alkyl-, heteroaryl-C₁-C₄ alkyl-, or heterocycyl-C₁-C₄ alkyl- is unsubstituted or substituted by one or more substituents independently selected from C₁-C₄ alkyl, C₁-C₄ haloalkyl and halogen.

In a another embodiment of the invention, R^{A} is —-CONHR¹. In a further embodiment of the invention, R^{A} is -CONHR¹ and R¹ is C₁-C₆ alkyl, aryl, heteroaryl, heterocycyl, aryl-C₁-C₄ alkyl-, heteroaryl-C₁-C₄ alkyl-, or heterocycyl-C₁-C₄ alkyl-, wherein said C₁-C₆ alkyl is optionally substituted with one ore more substituents independently selected from —NH₂, —N(C₁-C₄ alkyl)(C₁-C₄ alkyl), and —NH(C₁-C₄ alkyl), or said aryl, heteroaryl or heterocycyl or the aryl, heteroaryl or heterocycyl moiety of said aryl-C₁-C₄ alkyl-, heteroaryl-C₁-C₄ alkyl-, or heterocycyl-C₁-C₄ alkyl- is unsubstituted or substituted by one or more substituents independently selected from C₁-C₄ alkyl, C₁-C₄ haloalkyl and halogen. In yet a further embodiment of the invention, R^{A} is —CONHR¹ and R¹ is methyl, ethyl, phenyl, benzyl, phenethyl, N,N diethylaminopropyl, N-methyl-piperidinyl, piperidinyl-ethyl, pyrrolidinyl-butyl, morpholino-ethyl, or morpholino-propyl.

In another embodiment of the invention, R^{B} is —CONHR³, —SO₂R³, or —COC(R⁴R⁵)R³ where R³ is aryl or heteroaryl, wherein said aryl or heteroaryl is unsubstituted or substituted by one or more substituents independently selected from C₁-C₄ alkyl, C₁-C₄ haloalkyl, halogen, C₁-C₆ allyl, C₃-C₆ cycloalkyl, aryl, heteroaryl and heterocycyl.

In yet another embodiment of the invention, R^{B} is —CONHR³ or —SO₂R³ where R³ is aryl or heteroaryl, wherein said aryl or heteroaryl is unsubstituted or substituted by one or more substituents independently selected from C₁-C₄ alkyl, C₁-C₄ haloalkyl, halogen, C₁-C₆ alkyl, C₃-C₆ cycloalkyl, aryl, heteroaryl and heterocycyl.

In a yet another embodiment of the invention, R^{B} is —CONHR and R³ is aryl or heteroaryl, wherein said aryl or heteroaryl is unsubstituted or substituted by one or more substituents independently selected from C₁-C₄ alkyl, C₁-C₄ haloalkyl, halogen, C₁-C₆ alkyl, C₃-C₆ cycloalkyl, aryl, heteroaryl and heterocycyl.

In a yet another embodiment of the invention, R^{B} is —CONHR³ and R³ is substituted phenyl or substituted isoxazolyl, where said phenyl or isoxazolyl is substituted by one or more substituents independently selected from F, Cl, CF₃, or tert-butyl.

It is to be understood that reference to compounds of Formula I, II or III, herein refers to all compounds within the scope of Formula I, II or III, as defined above with respect to n, X, R, R^{B}, R¹, R² and R³, unless specifically limited otherwise.

For example one embodiment of this invention is directed to a compound of Formula I wherein: n is 1; R^{A} is —CONHR¹, —NHCOR¹, —NHSO₂R¹, where R¹ is C₁-C₆ alkyl, aryl, heteroaryl, heterocycyl, aryl-C₁-C₄ alkyl-, heteroaryl-C₁-C₄ alkyl-, or heterocycyl-C₁-C₄ alkyl-, wherein said C₁-C₆ alkyl is optionally substituted with one ore more substituents independently selected from —NH₂, —N(C₁-C₄ alkyl)(C₁-C₄ alkyl), and —NH(C₁-C₄ alkyl), or said aryl, heteroaryl or heterocycyl or the aryl, heteroaryl or heterocycyl moiety of said aryl-C₁-C₄ alkyl-, heteroaryl-C₁-C₄ alkyl-, or heterocycyl-C₁-C₄ alkyl- is unsubstituted or substituted by one or more substituents independently selected from C₁-C₄ alkyl, C₁-C₄ haloalkyl and halogen; X is O; and R^{B} is —CONHR³ or —SO₂NHR³; where R³ is aryl or heteroaryl, wherein said aryl or heteroaryl is unsubstituted or substituted by one or more substituents independently selected from C₁-C₄ alkyl, C₁-C₄ haloalkyl or halogen; or a salt, solvate, or physiologically functional derivative thereof.

In another embodiment of the compounds of Formula I, n is 1;R^{A} is —CONHR¹, and R¹ is methyl, ethyl, phenyl, benzyl, phenethyl, N,N diethylaminopropyl, N-methyl-piperidinyl, piperidinyl-ethyl, pyrrolidinyl-butyl, morpholino-ethyl, or morpholino-propyl; X is O; and R^{B} is —CONHR³, where R³ is substituted phenyl or substituted isoxazolyl, where said phenyl or isoxazolyl is substituted by one or more substituents independently selected from F, Cl, CF₃, or tert-butyl; or a salt, solvate, or physiologically functional derivative thereof.

Specific examples of compounds of the present invention include the following compounds:

5-(2-methylcarbamoyl-pyridin-4-yloxy)-2,3-dihydro-indole-l-carboxylic acid (4′-chloro-3′-tritluoromethyl-phenyl)-amide,

5-(2-methylcarbamoyl-pyridin-4-yloxy)-2,3-dihydro-indole-1-carboxylic acid (2′-fluoro-5′-trifluoromethyl-phenyl)-amide,

5-(2-methylcarbamoyl-pyridin-4-yloxy)-2,3-dihydro-indole-1-carboxylic acid (2′-fluoro-5′-trifluoromethyl-phenyl)-amide,

5-(2-hexylcarbamoyl-pyridin-4-yloxy)-2,3-dihydro-indole-1-carboxylic acid (3-trifluoromethyl-phenyl)-amide trifluoroacetate,

5-2-methylcarbamoyl-pyridin-4-yloxy)-2,3-dihydro-indole-1-carboxylic acid (4′-fluoro-3′-trifluoromethyl-phenyl)-amide,

5-(2-methylcarbamoyl-pyridin-4-yloxy)-2,3-dihydro-indole-1-carboxylic acid (3′-chlorophenyl)-amide,

5-(2-methylcarbamoyl-pyridin-4-yloxy)-2,3-dihydro-indole-1-carboxylic acid (bis-3′,5′-trifluoromethylphenyl)-amide,

5-(2-ethylcarbamoyl-pyridin-4-yloxy)-2,3-dihydro-indole-1-carboxylic acid (3-trifluoromethyl-phenyl)-amide,

5-(2-propylcarbamoyl-pyridin-4-yloxy)-2,3-dihydro-indole-1-carboxylic acid (3-trifluoromethyl-phenyl)-amide,

5-(2-phenylcarbamoyl-pyridin-4-yloxy)-2,3-dihydro-indole-1-carboxylic acid (3-trifluoromethyl-phenyl)-amide,

5-(2-benzylcarbamoyl-pyridin-4-yloxy-2,3-dihydro-indole-1-carboxylic acid (3-trifluoromethyl-phenyl)-amide,

5-(2-phenethylcarbamoyl-pyridin-4-yloxy)-2,3-dihydro-indole-1-carboxylic acid (3-trifluoromethyl-phenyl)-amide,

5-[2-(1-methyl-piperidin-4-ylcarbamoyl)-pyridinyloxy]-2,3-hydro-indole-1-carboxylic acid(3-trifuoromethyl-phenyl)-amide,

5-[2-(1-methyl-piperidin-4-ylcarbamoyl)-pyridin-4-yloxy]-2,3-dihydro-indole-1-carboxylic acid(3-trifuoromethyl-phenyl)-amide,

5-[2-(3-diethylamino-propylcarbamoyl)-pyridin-4-yloxy]-2,3-dihydro-indole-1-carboxylic acid (3-trifluoromethyl-phenyl)-amide,

5-[2-(3-morpholin-4-propylcarbamoyl)-pyridin-4-yloxy]-2,3-dihydro-indole-1-carboxylic acid (3-trifluoromethyl-phenyl)-amide,

5-[2-(2-piperidin-1-yl-ethylcarbamoyl)-pyridin-4-yloxy]-2,3-dihydro-indole-1-carboxylic acid (3-trifluoromethyl-phenyl)-amide,

5-[2-(4-pyrrolidin-1-yl-butylcarbamoyl)-pyridin-4-yloxy]-2,3-dihydro-indole-1-carboxylic acid (3-trifluoromethyl-phenyl)amide,

5-2-isopropylcarbamoyl-pyridin-4-yloxy)-2,3-dihydro-indole-1-carboxylic acid (3-trifluoromethyl-phenyl)-amide trifluoroacetate,

5-2-isopropylcarbamoyl-pyridin-4-yloxy)-2,3-dihydroindole-1-carboxylic acid (3-trifuoromethyl-phenyl)-amide trifluoroacetate,

5-[2-(2-methoxy-ethylcarbamoyl)-pyridin-4-yloxy]-2,3-dihydro-indole-1-carboxylic acid (3-trifluoromethyl-phenyl)-amide trifluoroacetate,

5-[2-(2-phenoxy-ethylcarbamoyl)-pyridin-4-yloxy]-2,3-dihydro-indole-1-carboxylic acid (3-trifluoromethyl-phenyl)-amide trifluoroacetate,

5-[2-(3-ethoxy-propylcarbamoyl)-pyridin-4-yloxy]-2,3-dihydro-indole-1-carboxylic acid (3-trifluoromethyl-phenyl)-amide trifluoroacetate,

5-[2-(3-isopropoxy-propylcarbamoyl)-pyridin-4-yloxy]-2,3-dihydro-indole-1-carboxylic acid (3-trifluoromethyl-phenyl)-amide trifluoroacetate, and

5-(2-hexylcarbamoyl-pyridin-4-yloxy)-2,3-dihydro-indole-1-carboxylic acid (3-trifluoromethyl-phenyl)-amide trirluoroacetate,

or a salt, solvate, or physiologically functional derivative thereof.

Typically, the salts of the present invention are pharmaceutically acceptable salts. The term “pharmaceutically acceptable salt” is intended to describe a salt that retains the biological effectiveness of the free acid or base of a specified compound and is not biologically or otherwise undesirable.

If an inventive compound is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycollates, tartrates mandelates, and sulfonates, such as xylenesulfonates, methanesulfonates, propanesulfonates, naphthalene-1-sulfonates and naphthalene-2-sulfonates.

If an inventive compound is an acid, a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Other salts, which are not pharmaceutically acceptable, may be useful in the preparation of compounds of this invention and these form a further aspect of the invention.

While it is possible that, for use in therapy, therapeutically effective amounts of a compound of Formula I, II or III, as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the Formula I, II or III and/or salts, solvates and/or physiological functional derivatives thereof, and one or more pharmaceutically acceptable carriers, diluents, or excipients. The compounds of the Formula I, II or III and salts, solvates and physiological functional derivatives thereof, are as described above. The carrier(s), diluent(s) or excipient(s) must be acceptable in the sense of being compatible with the other ingredients of the formulation and not deleterious to the recipient thereof in accordance with another aspect of the invention there is also provided a process for the preparation of a pharmaceutical formulation including admixing a compound of the Formula I, II or III, or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the Formula I, II or III, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

For example, pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffm, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticling to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating comprising a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of Formula I, II or III, and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of Formula I, I or III and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide—phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 1986, 3(6):318.

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior.;to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of Formula I, II or III for the treatment of neoplastic growth, for example colon or breast carcinoma, will generally be in the range of 0.1 to 100 mg/kg body weight of recipient (mammal) per day and more usually in the range of 1 to 10 mg/kg body weight per day. Thus, for a 70kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of Formula I, II or III per se. It is envisaged that similar dosages would be appropriate for treatment of the other conditions referred to herein.

The compounds of the present invention and their salts and solvates, and physiologically functional derivatives thereof, may be employed alone or in combination with other therapeutic agents for the treatment of the above-mentioned conditions. In particular, in anti-cancer therapy, combination with other chemotherapeutic, hormonal or antibody agents is envisaged as well as combination with surgical therapy and radiotherapy. Combination therapies according to the present invention thus comprise the administration of at least one compound of Formula I, II or III or a pharmaceutically acceptable salt or solvate thereof, or a physiologically functional derivative thereof, and the use of at least one other cancer treatment method. Preferably, combination therapies according to the present invention comprise the administration of at least one compound of Formula I, II or III or a pharmaceutically acceptable salt or solvate thereof, or a physiologically functional derivative thereof, and at least one other pharmaceutically active agent, preferably an anti-neoplastic agent. The compound(s) of Formula I, II or III and the other pharmaceutically active agent(s) may be administered together or separately and, when administered separately this may occur simultaneously or sequentially in any order. The amounts of the compound(s) of Formula L II or m and the other pharmaceutically active agent(s) and the relative timings of administration will be selected in order to achieve the desired combined therapeutic effect.

The compounds of the Formula I, II or III or salts, solvates, or physiologically functional derivatives thereof and at least one additional cancer treatment therapy may be employed in combination concomitantly or sequentially in any therapeutically appropriate combination with such other anti-cancer therapies. In one embodiment, the other anti-cancer therapy is at least one additional chemotherapeutic therapy including administration of at least one anti-neoplastic agent. The administration in combination of a compound of Formula I, II or III or salts, solvates, or physiologically functional derivatives thereof with other anti-neoplastic agents may be in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one anti-neoplastic agent is administered first and the other second or vice versa. Such sequential administration may be close in time or remote in time.

Anti-neoplastic agents may induce anti-neoplastic effects in a cell-cycle specific manner, i.e., are phase specific and act at a specific phase of the cell cycle, or bind DNA and act in a non cell-cycle specific manner, i.e., are non-cell cycle specific and operate by other mechanisms.

Anti-neoplastic agents useful in combination with the compounds and salts, solvates or physiologically functional derivatives thereof of Formula I, II or III include the following:

(1) cell cycle specific anti-neoplastic agents include, but are not limited to, diterpenoids such as paclitaxel and its analog docetaxel; vinca alkaloids such as vinblastine, vincristine, vindesine, and vinorelbine; epipodophyllotoxins such as etoposide and teniposide; fluoropyrimidines such as 5-fluorouracil and fluorodeoxyuridine; antimetabolites such as gemciabine, Fludarabine, methotrexate, cladrabine, cytarabine, mercaptopurine and thioguanine; and camptothecins such as 9-amino camptothecin, irinotecan, topotecan, and the various-optical forms of 7-(4methylpiperazino-methylene)-10,11-ethylenedioxy-20-camptothecin;.

(2) cytotoxic chemotherapeutic agents including, but not limited to, alkylating agents such as melphalan, chlorambucil, cyclophosphamide, mechlorethamine, hexamethylmelamine, busulfan, carmustine, lomustine, and dacarbazine; anti-tumor antibiotics such as beomycin, doxonibicin, daunomycin, epirubicin, idarubicin, mitoxantrone, mitomycin-C, dacttinomycin and mithramycin; and platinum coordination complexes such as cisplatin, carboplatin, and oxaliplatin; and

(3) other chemotherapeutic agents including, but not limited to, anti-estrogens such as tamoxifen, toremifene, raloxifene, droloxifene and iodoxyfene; progestrogens such as megestrol acetate; aromatase inhibitors such as anastrozole, letrazole, vorazole, and exemestane; antiandrogens such as flutamide, nilutamnide, bicalutamide, and cyproterone acetate; glucocorticoids such as prednisone and decadron; LHRH agonists and antagonists such as goserelin acetate and luprolide; testosterone 5α-dihydroreductase inhibitors such as dutasteride, finasteride; metalloproteinase inhibitors such as marimastat; antiprogestogens; other biologic agents such as L-asparaginase; urokinase plasminogen activator receptor function inhibitors; small molecule and antibody growth factor function inhibitors such as inhibitors of the functions of hepatocyte growth factor; erb-B2, erb-B4, epidermal growth factor receptor (EGFR), platelet derived growth factor receptor (PDGFR), tyrosone kinases such as, EphB₄, c-kit and bcr/abl), vascular endothelial growth factor receptor (VEGFR), and TIE-2 (other than those VEGFR and TIE-2 inhibitors described in the present invention); and other kinase inhibitors such as inhibitors of CDK2 and CDK4; and other antitumor agents such as thalidomide, immunoconjugates, cytokines, such as IL-2, IFN alpha and beta, tumor vaccines including dendritic cell vaccines, all cyclooxygenase inhibitors and radiation sensitizers such as temazolamide

The compounds of Formula I, II or III and salts, solvates and physiological functional derivatives thereof, are active as inhibitors of at least one of the protein kinases TIE-2, VEGFR-2, VEGFR-3 and Raf.

The present invention thus also provides compounds of Formula I, I or III and pharmaceutically acceptable salts or solvates thereof, or physiologically functional derivatives thereof, for use in medical therapy, and particularly in the treatment of disorders mediated by at least one of inappropriate TIE-2, VEGFR-2, VEGFR-3 and Raf kinase activity.

The inappropriate TIE-2, VEGFR-2, VEGFR-3 and/or Raf kinase activity referred to herein is any TIE-2, VEGFR-2, VEGFR-3 and/or Raf kinase activity that deviates from the normal TIE-2, VEGFR-2, VEGFR-3 and/or Raf kinase activity expected in a particular mammalian subject. Inappropriate TIE-2, VEGFR-2, VEGFR-3 and/or Raf kinase activity may take the form of, for instance, an abnormal increase in activity, or an aberration in the timing and or control of TIE-2, VEGFR-2, VEGFR-3 and/or Raf kinase activity. Such inappropriate activity may result then, for example, from overexpression or mutation of the protein kinase leading to inappropriate or uncontrolled activation. Furthermore, it is also understood that unwanted TIE-2, VEGFR-2, VEGFR-3 kinase and/or Raf activity may reside in an abnormal source, such as a malignancy. That is, the level of TIE-2, VEGFR-2, VEGFR-3 and/or Raf kinase activity does not have to be abnormal to be considered inappropriate, rather the activity derives from an abnormal source.

In a like manner, the inappropriate angiogenesis referred to herein is any angiogenic activity that deviates from the normal angiogenic activity expected in a particular mammalian subject. Inappropriate angiogenesis may take the form of, for instance, an abnormal increase in activity, or an aberration in the timing and or control of angiogenic activity. Such inappropriate activity may result then, for example, from overexpression or mutation of a protein kinase leading to inappropriate or uncontrolled activation. Furthermore, it is also understood that unwanted angiogenic activity may reside in an abnormal source, such as a malignancy. That is, the level of angiogenic activity does not have to be abnormal to be considered inappropriate, rather the activity derives from an abnormal source.

The present invention is directed to methods of regulating, modulating, or inhibiting TIE-2, VEGFR-2, VEGFR-3 and/or Raf kinase for the prevention and/or treatment of disorders related to inappropriate TIE-2, VEGFR-2, VEGFR-3 and/or Raf activity.

In particular, the compounds of the present invention are useful in the treatment of susceptible forms of cancer, including tumor growth and metastasis. Furthermore, the compounds of the present invention can be used to provide additive or synergistic effects with certain existing cancer chemotherapies, and/or be used to restore effectiveness of certain existing cancer chemotherapies and radiation.

The compounds of the present invention may be also useful in the treatment of one or more diseases afflicting mammals which are characterized by cellular proliferation in the area of disorders associated with neo-vascularization and/or vascular permeability including blood vessel proliferative disorders including arthritis and restenosis; fibrotic disorders including hepatic cirrhosis: and atherosclerosis; mesangial cell proliferative disorders including glomerulonephritis, diabetic nephropathy, malignant nephrosclerosis, thrombotic microangiopathy syndromes, organ transplant rejection and glomerulopathies; and metabolic disorders including psoriasis, diabetes mellitus, chronic wound healing, inflammatory diseases (e.g., rheumatoid arthritis), stroke and neurodegenerative diseases; also diabetic retinopathy; macular degeneration; other diseases characterized by ocular neovascularization; and diseases characterized by hemangiomas.

A further aspect of the invention provides a method of treatment of a mammal suffering from a disorder mediated by at least one of inappropriate TIE-2, VEGFR-2, VEGFR-3 and Raf activity, which includes administering to said subject an effective amount of a compound of Formula I, II or III or a pharmaceutically acceptable salt, solvate, or a physiologically functional derivative thereof. In a preferred embodiment, the disorder is cancer, e.g., malignant tumors. Another aspect of the invention also provides such a method wherein the disorder is a disease afflicting mammals which are characterized by cellular proliferation in the area of disorders associated with neo-vascularization and/or vascular permeability, including those disclosed herein.

A further aspect of the invention provides a method of treatment of a mammal suffering from cancer which includes administering to said subject an effective amount of a compound of Formula I, II or III or a pharmaceutically acceptable salt or solvate thereof, or a physiologically functional derivative thereof.

A further aspect of the present invention provides the use of a compound of Formula I, II or III, or a pharmaceutically acceptable salt or solvate thereof, or a physiologically functional derivative thereof, in the preparation of a medicament for the treatment of a disorder characterized by at least one of inappropriate TIE-2, VEGFR-2 VEGFR-3 and Raf kinase activity. In a preferred embodiment, the disorder is cancer, e.g., malignant tumors. Another aspect of the invention also provides such a use wherein the disorder is a disease afflicting mammals which are characterized by cellular proliferation in the area of disorders associated with neo-vascularization and/or vascular permeability, including those disclosed herein.

A further aspect of the present invention provides the use of a compound of Formula I, II or III, or a pharmaceutically acceptable salt or solvate thereof, or a physiologically functional derivative thereof, in the preparation of a medicament for the treatment of cancer,-e.g., malignant tumors.

The mammal requiring treatment with a compound of the present invention is typically a human being.

In another embodiment, therapeutically effective amounts of (a) the compounds of Formula I, II or m or salts, solvates or physiologically derived derivatives thereof and (b) agents which inhibit kinase signaling may be administered in combination to a mammal for treatment of a disorder mediated by at least one of inappropriate TIE2, VEGFR-2, VEGFR-3 and Raf kinase activity, for instance in the treatment of cancer, e.g., malignant tumors. Such kinase signaling receptors include, for example, EGFR, PDGFR, erbB2, erbB4, VEGFR, TIE-2, Raf, Akt, PI₃K, and mTor. Oncogenic kinase signaling receptors and agents that inhibit their kinase function are described, for instance, in Kath, John C., Exp. Opin. Ther. Patenzts (2000) 10(6): 803-818 and in Blume-Jensen, Peter, Nature (2001)411:355.

The compounds of the Formula I, II or III or salts, solvates, or physiologically functional derivatives thereof and the agent for inhibiting growth factor receptor function may be employed in combination concomitantly or sequentially in any therapeutically appropriate combination. The combination may be employed in combination in accordance with the invention by administration concomitantly in (1) a unitary pharmaceutical composition including both compounds, or (2) separate pharmaceutical compositions each including one of the compounds. Alternatively, the combination may be administered separately in a sequential manner wherein one is administered first and the other second or vice versa. Such sequential administration may be close in time or remote in time.

In another aspect of the present invention, there is provided a method of treating a disorder in a mammal, said disorder being mediated by inappropriate angiogenesis, including: administering to said mammal a therapeutically effective amount of a compound of Formula I II or III or a salt, solvate or physiologically functional derivative thereof. In one embodiment, the inappropriate angiogenic activity is due to at least one of inappropriate VEGFR1, VEGFR2, VEGFR3, or TIE-2 activity. In another embodiment, the inappropriate angiogenesis is due to at least one of inappropriate VEGFR-2, VEGFR-3, and TIE-2 Kinase activity. In a preferred embodiment, the inappropriate angiogenic activity is due to at least one of inappropriate VEGFR-2 and TIE-2 kinase activity. In a further embodiment, the method further includes administering a therapeutically effective amount of a VEGFR2 inhibitor along with the compounds of Formula 1, I[ or m or salts, solvates or physiologically functional derivatives thereof. Preferably the disorder is cancer; e.g., malignant tumors. This aspect of the invention also provides such methods wherein the disorder is a disease afflicting mammals which are characterized by cellular proliferation in the area of disorders associated with neo-vascularization and/or vascular permeability, including those disclosed herein.

In another aspect of the present invention, there is provided the use of a compound of Formula I, I or m, or a salt, solvate or physiologically functional derivative thereof in the preparation of a medicament for use in treating a disorder in a mammal, said disorder being characterized by inappropriate angiogenesis. In one embodiment, the inappropriate angiogenic activity is due to at least one of inappropriate VEGFR1, VEGFR2, VEGFR3 or TIE-2 activity. In another embodiment, the inappropriate angiogenesis is due to at least one of inappropriate VEGFR-2, VEGFR-3, and TIE-2 kinase activity. In a preferred embodiment, the inappropriate angiogenic activity is due to at least one of inappropriate VEGFR-2 and TIE-2 Kinase activity. In a further embodiment, the use further includes use of a VEGFR2 inhibitor to prepare said medicament. Preferably the disorder is cancer, e.g., malignant tumors. This aspect of the invention also provides such uses wherein the disorder is a disease afflicting mammals which are characterized by cellular proliferation in the area of disorders associated with neo-vascularization and/or vascular permeability, including those disclosed herein. The combination of a compound of Formula I, II or III or salts, solvates, or physiologically functional derivatives thereof with a VEGFR2 inhibitor may be employed in combination in accordance with the invention by administration concomitantly in (1) a unitary pharmaceutical composition including both compounds, or (2) separate pharmaceutical compositions each including one of the compounds. Alternatively, the combination may be administered separately in a sequential manier wherein one is administered first and the other second or vice versa. Such sequential administration may be close in time or remote in time.

A further aspect of the invention provides a method of treatment of a mammal suffering from a disorder mediated by mediated by inappropriate Raf kinase activity, which includes administering to said subject an effective amount of a compound of Formula I, II or III or a pharmaceutically acceptable salt, solvate, or a physiologically functional derivative thereof. Raf protein kinases are key components of signal transduction pathways by which specific extracellular stimuli elicit precise cellular responses in mammalian cells Activated cell surface receptors activate ras/rap proteins at the inner aspect of the plasma membrane which in turn recruit and activate Raf proteins. Activated Raf proteins phosphorylate and activate the intracellular protein kinases MEK1 and MEK2. In turn, activated MEKs catalyse phosphorylation and activation of p42/p44 mitogen-activated protein kinase (MAPK). A variety of cytoplasmic and nuclear substrates of activated MAPK are known which directly or indirectly contribute to the cellular response to environmental change. Three distinct genes have been identified in mammals that encode Raf proteins; A-Raf, B-Raf and C-Raf (also known as Raf-1) and isoformic variants that result from differential splicing of mRNA are known.

Inhibitors of Raf kinases have been suggested for use in disruption of tumor cell growth and hence in the treatment of cancers, e.g., melanoma, histiocytic lymphoma, lung adenocarcinoma, colorectal, ovarian, and small cell lung cancer and pancreatic and breast carcinoma;

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Examples.

Compounds of general Formula I, II or III may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula I, II or III. Those skilled in the art will recognize if a stereocenter exists in compounds of Formula I, II or III. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a-starting material may be effected by any suitable method known in the art. See for example, Stereochemistry of Organic Compounds by E. L. Eliel, S. H. Wilen, and L. N. Mander (Wiley-Interscience, 1994).

Compounds of Formula I, II or III can be prepared according to the synthetic sequences illustrated in Schemes 1 and 2, which shows general routes for the synthesis of the targeted 5-(2-methylcarbamoyl-pyridin-4-yloxy)-2,3-dihydro-indole derivatives. Specific details of synthetic routes according to Scheme 1 are shown in the Examples, which include the preparation of 5-(2-methylcarbamoyl-pyridin-4-yloxy)-2,3-dihydro-indole-1-carboxylic acid amide. All of the compounds specifically described herein may be prepared according to the procedures described in Examples 1-8 hereinbelow.

A synthesis of 5-(2-methylcarbamoyl-pyridin-4-yloxy)-2,3-dihydro-indole derivatives may be achieved by the route shown in Scheme 1. This synthesis uses an appropriately substituted pyridinyl chloride (ii), which is here exemplified with, but should not be seen as limited to, a methylcarbamoyl pyridyl chloride. Thus, a picolinic acid is treated with thionyl chloride in the presence of sodium bromide to give the intermediate 4-chloropyridine-2-carbonyl chloride, followed by treatment with the appropriate amine. The pyridinyl chloride (ii) is reacted with 1-acetyl-2,3-dihydro-5-hydroxyindole in the presence of sodium t-butoxide to generate the N-acetyl-diaryl ether, which upon treatment with acid provides the corresponding diaryl ether (iii). Treatment of (iii) with an appropriately substituted isocyanate gives the corresponding urea (iv); treatment of (iii) with an appropriately substituted acid in conjunction with standard peptide coupling reagents or appropriately substituted acid chloride gives the amides (v and vi); treatment of (iii) with an appropriately substituted sulphonyl chloride to give sulphonamide (vii).

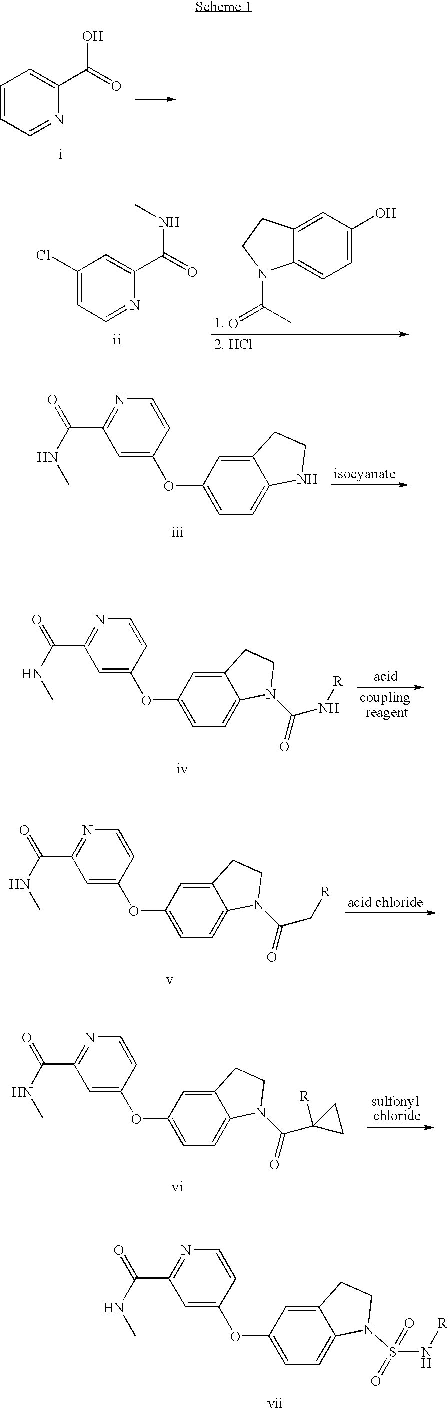

An effective synthesis of 5-(2-methylcarbamoyl-pyridin-4-ylamino)-2,3-dihydro-indole derivatives may be achieved by the route shown in Scheme 2. This synthesis uses an appropriately substituted pyridinyl chloride (ii), which is here exemplified with, but should not be seen as limited to, a methylcarbamoyl pyridyl chloride. The pyridinyl chloride (ii), described in Scheme 1, is reacted with 1-acetyl-2,3-dihydro-5-aminoindole in the presence of acid, such as ethereal hydrogen chloride, to generate the N-acetyl-diaryl amine salt which undergoes alkylation with standard conditions such as, but not limited, to methyl iodide in the presence of a base such as potassium carbonate in a solvent like DMF to give, after treatment with aqueous acid, the corresponding diaryl amine (viii). Treatment of (viii) with an appropriately substituted isocyanate gives the corresponding urea (ix); treatment of (viii) with an appropriately substituted acid in conjunction with standard peptide coupling reagents or appropriately substituted acid chloride gives the amides (x and xi); and treatment of (viii) with an appropriately substituted sulphonyl chloride gives sulphonamide (xii).

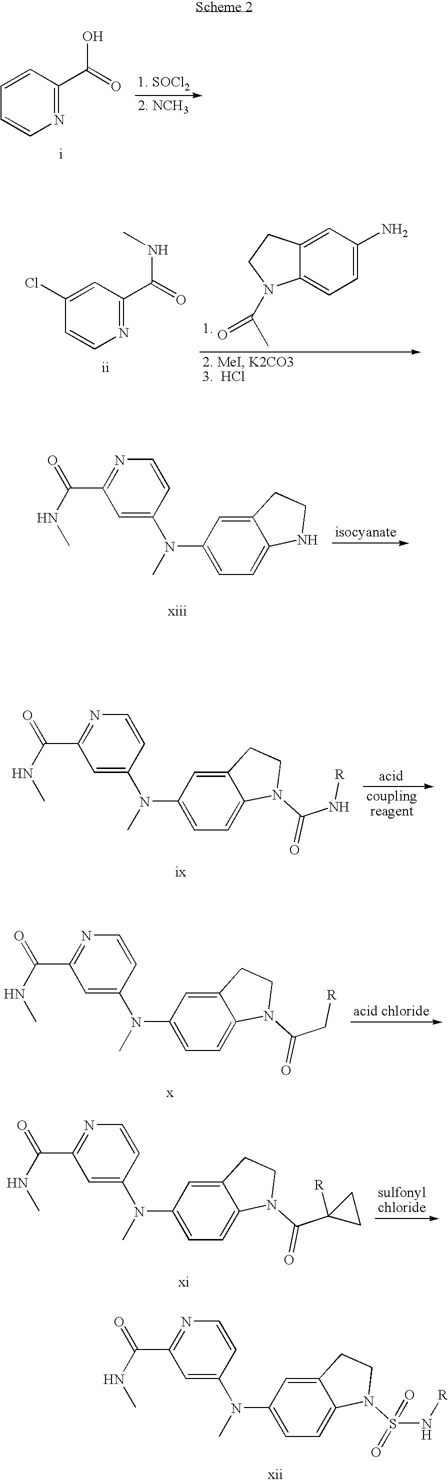

### EXAMPLES

Certain embodiments of the present invention will now be illustrated by way of example only. The physical data given for the compounds exemplified is consistent with the assigned structure of those compounds.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification: g (grams); mg (milligrams); L (liters); mL (milliliters); μL (microliters); psi (pounds per square inch); M (molar); mM (millimolar); i. v. (intravenous); Hz (Hertz); MHz (megahertz); mol (moles); mmol (millimoles); rt (room temperature); min (minutes); h (hours); mp (melting point); TLC (thin layer chromatography); Tᵣ (retention time); RP (reverse phase); MeOH (methanol); i-PrOH (isopropanol); TEA (triethylamine); TFA (trifluoroacetic acid); TFAA (trifluoroacetic anhydride); THF (tetrahydrofuran); DMSO (dimethylsulfoxide); AcOEt (ethyl acetate); DME (1,2-dimethoxyethane); DCM (dichloromethane); DCE (dichloroethane); DMF (N,N-dimethylformamide); DMPU (N,N′-dimethylpropyleneurea); CDI (1,1-carbonyldiimidazole); IBCF (isobutyl chloroformate); HOAc (acetic acid); HOSu (N-hydroxysuccinimide); HOBT (1-hydroxybenzotriazole); mCPBA (meta-chloroperbenzoic acid; EDC (ethylcarbodiimide hydrochloride); BOC (tert-butyloxycarbonyl); FMOC (9-fluorenylmethoxycarbonyl); DCC (dicyclohexylcarbodiimide); CBZ (benzyloxycarbonyl); Ac (acetyl); atm (atmosphere); TMSE (2-(trimethylsilyl)ethyl); TMS (trimethylsilyl); TIPS (triisopropylsilyl); TBS (t-butyldimethylsilyl); DMAP (4-dimethylaminopyridine); BSA (bovine serum albumin) ATP (adenosine triphosphate); HRP (horseradish peroxidase); DMEM (Dulbecco's modified Eagle medium); HPLC (high pressure liquid chromatography); BOP (bis(2-oxo-3-oxazolidinyl)phosphinic chloride); TBAF (tetra-n-butylammonium fluoride); HBTU (O-Benzotriazole-1-yl-N,N,N′,N′-tetramethyluronium hexafluorophosphate). HEPES (4-(2-hydroxyethyl)-1-piperazine ethane sulfonic acid); DPPA (diphenylphosphoryl azide); fHNO₃ (fumed HNO₃); and EDTA (ethylenediaminetetraacetic acid).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR (hereinafter also “NMR”) spectra were recorded on a Varian VXR-300, a Varian Unity-300, a Varian Unity-400 instrument, a Brucker AVANCE-400, a General Electric QE-300, or a Bruker AM 400 spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Mass spectra were run on an open access LC/MS system using electrospray ionization. LC conditions: 4.5% to 90% CH₃CN (0.02% TFA) in 3.2 min with a 0.4 min hold and 1.4 min re-equilibration; detection by MS, UV at 214 nm, and a light scattering detector (ELS). Column: 1×40 mm Aquasil (C_{1 8}).

For analytical hplc; ca 0.05 mg of the reaction mixtures were injected in 5 uL of DMSO onto a 4.6×150 mm I. D. Zorbax Eclipse XDB-C₁I column at 3 mL/min with a 10 min gradient from 5% CH₃CN (0.1% TFA) to 95% CH₃CN (0.1% TFA) in H₂O (0.1% TFA).

For preparative (prep) hplc; ca 50 mg of the final products were injected in 500 uL of DMSO onto a 50×20 mm I. D. YMC CombiPrep ODS-A column at 20 mL/min with a 10 min gradient from 10% CH₃CN (0.1% TFA) to 90% CH₃CN (0.1% TFA) in H₂O (0.1% TFA) and a 2 min hold. Flash chromatography was run over Merck Silica gel 60 (230-400 mesh).

Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution.

### Example 1

### 5-(2-Methylcarbamoyl-pyridin-4-yloxy)-2,3-dihydro-indole-1-carboxylic acid (2′-fluoro-5′-trifluoromethyl-phenyl)-amide

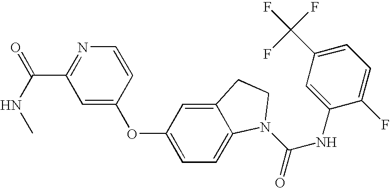

a) 4-Chloro-pyridine-2-carboxylic acid methylamide. Picolinic acid (6.6 g, 50 mmol) was dissolved in thionyl chloride (30 mL) and potassium bromide was added (595 mg, 5 mmol). The mixture was kept at reflux overnight then the resulting red solution was concentrated under reduced pressure. The resultant red oil was dissolved in toluene, filtered to remove KBr, and concentrated under reduced pressure (×3). The red oil was then dissolved in THF (100 mL) and added dropwise to a stirred solution of 2M methylamine in THF (100 mL, 200 mmol) at rt. The reaction was stirred overnight at rt. The THF was removed under reduced pressure, and the residue was partitioned between ethyl acetate and 5% sodium bicarbonate solution. The layers were separated and the organic layer washed with water (×3) and brine. After drying (MgSO₄) concentration gave a red oil (6.6 g) which was purified by chromatography on silica gel eluting with a gradient of Ethyl acetate/hexane to afford the title compound as an oil which crystallized on standing. (4.3 g, 50%) LC-MS (m/e)=171.0 [M+H]⁺. Retention time=1.29 min.

b) 4-(1-Acetyl-2,3-dihydro-1-H-indol-5-yloxy)-pyridine-2-carboxylic acid methylamide. 1-Acetyl-2,3-dihydro-5-hydroxyindole (700 mg, 4 mmol) which may be prepared by the method as described by Rickard Hunt in J.Chem.Soc.C, 344 (1966), was dissolved in DMF (4 ml) and treated at rt for 5 min with potassium t-butoxide (500 mg, 1.1 eq) followed by addition of 4-Chloro-pyridine-2-carboxylic acid methylamide (680 mg, 4 mmol) and potassium carbonate (550 mg). This mixture was microwaved for 10 min at 160° in a Personal Chemistry synthesizer. Hplc showed incomplete reaction, so the mixture was diluted to a volume a 10 mL (DMF), treated with additional of 4-Chloro-pyridine-2-carboxylic acid methylamide (150 mg, 0.88 mmol) and potassium t-butoxide (100 mg), and microwaved for 20 min at 160°. The mixture was partitioned between ethyl acetate and water and the aqueous extracted with EtOAc (×4). The combined extracts were washed with water (×3) and brine, then dried (MgSO₄) and evaporated under reduced pressure to afford the title compound as a off-white solid after trituration with Et₂O/hexane. (1.042 g, 96.5%) LC-MS (m/e)=312.2 [M+H]⁺. Retention time=1.62 min. Hplc 93%.

c) 4-(2,3-Dihydro-1-H-indol-5-yloxy)-pyridine-2-carboxylic acid methylamide. 4-(1-Acetyl-2,3dihydro-1-H-indol-5-yloxy)-pyridine-2-carboxylic acid methylamide (999 mg, 3.2 mmol) in 6N HCl (15 ml, 5 ml in each of 3 vials) was placed in the microwave reactor and heated for 15 min at 130°. The aqueous solution was made basic to pH 9 with 5% NaHCO₃ and extracted with EtOAc (×4). The combined extracts were washed with brine, dried (MgSO₄) and evaporated under reduced pressure to afford the title compound as a white foam (0.480 g, 56%) after flash chromatography eluting with 30-70% EtOAc/Hexane. LC-MS (m/e)=270.4 [M+H]⁺. Retention time=1.15 min. Hplc 92%.

d) 5-(2-Methylcarbamoyl-pyridin-4-yloxy)-2,3-dihydro-indole-1-carboxylic acid (2′-fluoro-5′-trifluoromethyl-phenyl)-amide. To a solution of 2-fluoro-5-trifluoromethyl-phenylisocyanate (24.6 mg, 0.12 mmol) in dichloromethane (0.5 mL) was added 4-(2,3-Dihydro-1-H-indol-5-yloxy)-pyridine-2-carboxylic acid methylamide (32 mg, 0.12 mmol) at 0°. The reaction was allowed to warm to rt after 30 min at 0° and stirred overnight at rt. Hplc confirmed complete reaction. A white precipitate, which had formed, was removed by filtration after dilution with Et₂O to provide the title compound as a white solid (34 mg, 60%). LC-MS (m/e)=475.2 [M+H]⁺. Retention time=2.22 min. Hplc 100%. ¹H NMR(400 MHz, CDCl₃) 58.52-8.48 (m, 1H), 8.40 (d, J=7.6 Hz, 1H), 8.120 (br s, 1H) 8.018 (d, J=9.3 Hz, 1H), 7.71 (d, J=2.5 Hz, 1H), 7.31-7.26 (m, 2H), 7.02-6.97 (m, 3 H), 6.83 (d, J=3.8 Hz, 1H), 4.23 (t, 2H, J=8.5 Hz), 3.33 (t, 2H, J=8.5 Hz), 3.03 (d, J=5.1 Hz, 3H).

### Example 2

### 5-(2-Methylcarbamoyl-pyridin-4yloxy)-2,3-dihydro-indole-1-carboxylic acid(2′-chloro-5′-trifluoromethyl-phenyl)-amide

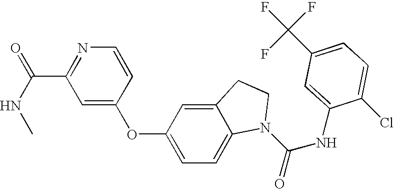

Following the procedure of Example 1(d), except substituting (2-chloro-5-trifluoromethyl-phenylisocyanate for 2-fluoro-5-trifluoromethyl-phenylisocyanate, the title compound was prepared as a white solid (50 mg, 85%). LC-MS (m/e)=491.0 [M+H]⁺. Retention time=2.44 min. Hplc 100%. ¹H NMR(400 MHz, CDCl₃) δ8.75 (d, J=2.2 1H), 8.40 (d, J=5.6 Hz, 1H), 8.168 (br s, 1H, NH) 8.07 (d, J=8.4 Hz, 1H), 7.72 (d, J=2.5 Hz, 1H), 7.52 (d, J=8.4 Hz, 1H), 7.31-7.26 (m, 2H), 7.02-6.97 (m, 3H), 4.26 (t, 2H, J=8.5 Hz), 3.34 (t, 2H, J=8.5 Hz), 3.03 (d, J=5.1 Hz, 3H).

### Example 3

### 5-(2-Methylcarbamoyl-pyridin-4-yloxy)-2,3-dihydro-indole-1-carboxylic acid(3′-trifluoromethyl-phenyl)-amide

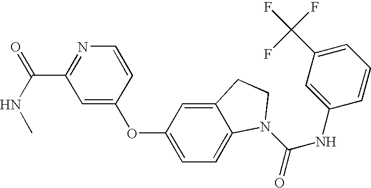

Following the procedure of Example 1(d), except substituting (3-trifluoromethyl-phenylisocyanate for 2-fluoro-5-trifluoromethyl-phenylisocyanate, the title compound was prepared as a white solid (45.6 mg, 83%). LC-MS (nm/e)=457.20 [M+H]⁺. Retention time=2.29 min. Hplc 98.5%. ¹H NMR(400 MHz, CDCl₃) δ8.40 (d, J=5.6 Hz, 1H), 8.18 (br s, 1H, NH), 8.00 (d, J=9.4 Hz, 1H), 7.76-7.68 (m, 3H), 7.46 (t, J=7.8 Hz, 1H), 7.35(d, J=7.8 Hz, 1H), 7.03-6.88 (m, 4H), 4.17 (t, 2H, J=8.5 Hz), 3.28 (t, 2H, J=8.5 Hz), 3.03 (d, J=5.1 Hz, 3H).

### Example 4

### 5-(2-Methylcarbamoyl-pyridin-4-yloxy)-2,3-dihydro-indole-1-carboxylic acid(4′-fluoro-3′-trifluoromethyl-phenyl)-amide

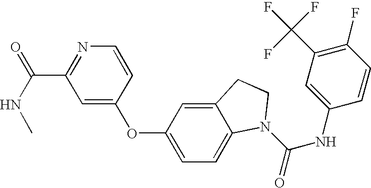

Following the procedure of Example 1(d), except substituting (4-fluoro-3-trifluoromethyl-phenylisocyanate for 2-fluoro-5-trifluoromethyl-phenylisocyanate, the title compound was prepared as a white solid (41.1 mg, 72%). LC-MS (m/e)=475.2 [M+H]⁺. Retention time=2.27 min. Hplc 100%. ¹H NMR(400 MHz, CDCl₃) δ8.40 (d, J=5.6 Hz, 1H), 8.20 (br s, 1H, NM) 8.00 (d, J=9.1 Hz, 1H), 7.17 (t, J=9.1 Hz, 1H), 7.03 (dd, J=5.6 Hz and J=2.6 Hz, 1H), 6.95-6.89 (m, 3H), 4.15 (t, J=8.5 Hz, 2H), 3.28 (t, J=8.5 Hz, 2H), 3.03 (d, J=5.1 Hz, 3H).

### Example 5

5-(2-Methylcarbamoyl-pyridin-4-yloxy)-2,3-dihydro-indole-1-carboxylic acid(3′-chlorophenyl)-amide

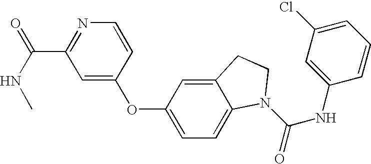

Following the procedure of Example l(d), except substituting (3-chloro-phenylisocyanate for 2-fluoro-5-trifluoromethyl-phenylisocyanate, the title compound was prepared as a white solid (39.7 mg, 78%). LC-MS (m/e)=423.0 [M+H]⁺. Retention time=2.17 min. Hplc 97%. ¹H NMR(400 MHz, CDCl₃) δ8.40 (d, J=5.6 Hz, 1H), 8.25 (br s, 1H, NH), 8.00 (d, J=9.4 Hz, 1H), 7.71 (d, J=2.4 Hz, 1H), 7.60 (t, J=2.1 Hz, 1H), 7.355-7.241(m, 3H), 7.09 (d, J=0.9 Hz, 1H), 4.17 (t, 2H, J=8.5 Hz), 3.28 (t, 2H, J=8.5 Hz), 3.03 (d, J=5.1 Hz, 3H).

### Example 6

### 5-(2-Methylcarbamoyl-pyridin-4-yloxy)-2,3-dihydro-indole-1-carboxylic acid(bis-3′,5′-trifluoromethylphenyl)-amide

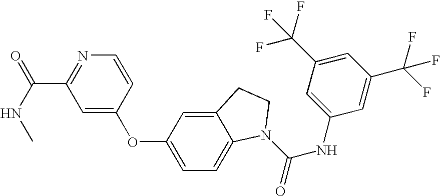

Following the procedure of Example 1(d), except substituting (bis-3,5-trifluoromethylphenyl-isocyanate for 2-fluoro-5-trifluoromethyl-phenylisocyanate, the title compound was prepared as a white solid (45.6 mg, 72%). LC-MS (m/e)=525.2 [M+H]⁺. Retention time=2.54 min. Hplc 100%. ¹H NMR(400 MHz, CDCl₃) δ8.40 (d, J=5.6 Hz, 1H), 8.26 (br s, 1H, NH), 8.010 (m, 3H), 7.66 (d, J=2.5 Hz, 1H), 7.57 (s, 1H), 7.188 (s, 1H), 7.05 (dd, J=5.6 Hz, J=2.6 Hz 1H), 4.17 (t, 2H, J=8.5 Hz), 3.29 (t, 2H, J=8.5 Hz), 3.03 (d, J=5.1 Hz, 3H).

### Example 7

### 5-(2-Methylcarbamoyl-pyridin-4-yloxy)-2,3-dihydro-indole-1-carboxylic acid(5-tert-butyl-isoxazol-3-yl)-amide trifluoroacetate

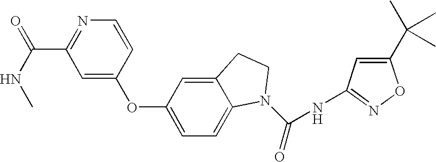

The compound of 1-Acetyl-2,3-dihydro-5-hydroxyindole (54 mg g, 2 mmol) was dissolved in DMF (4 mL) and treated with Hunig's base (72 uL, 4 mmol) and (5-t-butyl-isoxazol-3-yl)-carbamic acid phenyl ester (78 mg, 3 mmol), then microwaved at 190° for 800 sec. The crude reaction mixture was chromatographed on reverse phase column eluting 5%-95% acetonitrile/0.1% TFA water. The material isolated from this chromatography was then chromatographed using a different C₁₈ reverse phase column (45 mg) and finally recrystallized from acetonitrile/water to give white crystals. (20 mg, 23 %) LC-MS (m/e)=436.4 [M+H]⁺. Retention time=2.11 min. Hplc 93%. ¹H NMR(400 MHz, CD₃OD) δ8.50 (d, J=5.86 Hz, 1H), 8.04 (d, J=8.7 Hz 1H), 7.65 (d, J=2.4 Hz, 1H), 7.2-6.9 (m, 3H), 7.188 (s, 1H), 6.554 (s, 1H), 4.17 (t, J=8.5 Hz, 2H), 3.3 (t, 2H), 2.96 (s, 3H) 1.3 (s, 9H).

### Example 8

### 5-(2-Methylcarbamoyl-pyridin-4-yloxy)-2,3-dihydro-indole-1-carboxylic acid(4′-chloro-3′-trifluoromethyl-phenyl)-amide

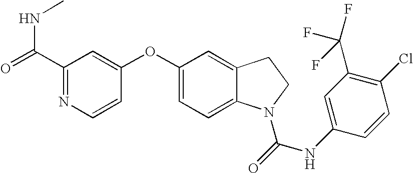

Following the procedure of Example 1(d), except substituting (4-chloro-3-trifluoromethyl-phenylisocyanate for 2-fluoro-5-trifluoromethyl-phenylisocyanate, the title compound was prepared as a white solid (850 mg, 86%). LC-MS (m/e)=491.04 [M+H]⁺. Retention time=2.44 min. Hplc 94%. ¹H NMR(400 MHz, CDCl₃) δ88.37 (d, J=5.6 Hz, 1H) 8.015 (br s, 1H, NH), 7.97 (m, 1H) 7.71 (d, J=2.5 Hz, 1H), 7.70 (dd, J=2.5 Hz, J=8.8 Hz, 1H), 7.63 (d, J=2.5 Hz, 1H), 7.452 (d, J=8.8 Hz, 1H), 6.37-6.922 (m, 3H), 6.71 (s, 1H), 4.14 (t, 2H, J=8.4 Hz), 3.26 (t, 2H, J=8.4 Hz), 3.01 (d, J=5.2 Hz, 3H).

### Examples 9-31

Following the procedure of Example 1(d), except substituting the appropriate isocyanate for 2-fluoro-5-trifluoromethyl-phenylisocyanate, the following title compounds were prepared:

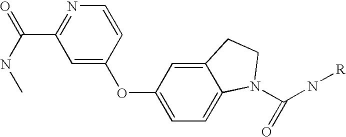

| | | | LC Mass |
|---|---|---|---|
| | | | Spectra |
| Example | | | [M + H] @ |
| # | R | Compound Name | ret time |
| 9 | 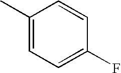 | 5-(2-Methylcarbamoyl-pyridin-4-yloxy)-2,3- dihydro-indole-1-carboxylic acid (4'-fluorophenyl)-amide | 407.2 @2.00 min |
| 10 | 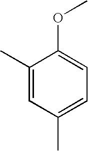 | 5-(2-Methylcarbamoyl-pyridin-4-yloxy)-2,3- dihydro-indole-1-carboxylic acid (2-methoxy-5-methyl-phenyl)-amide | 433.4 @2.24 min |
| 11 | 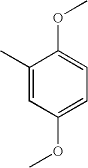 | 5-(2-Methylcarbamoyl-pyridin-4-yloxy)-2,3- dihydro-indole-1-carboxylic acid (2,5-dimethoxy-phenyl)-amide | 448.8 @2.15 min |
| 12 | 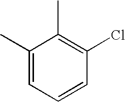 | 5-(2-Methylcarbamoyl-pyridin-4-yloxy)-2,3- dihydro-indole-1-carboxylic acid 3-chloro-2-methyl-phenyl)-amide | 436.6 @2.10 min |
| 13 | 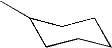 | 5-(2-Methylcarbamoyl-pyridin-4-yloxy)-2,3- dihydro-indole-1-carboxylic acid cyclohexylamide | 395.2 @2.02 min |
| 14 | 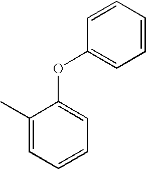 | 5-(2-Methylcarbamoyl-pyridin-4-yloxy)-2,3- dihydro-indole-1-carboxylic acid (2-phenoxy-pbenyl)-amide | 481.0 @2.42 min |
| 15 | 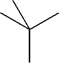 | 5-(2-Methylcarbamoyl-pyridin-4-yloxy)-2,3- dihydro-indole-1-carboxylic acid tert-butylamide | 369.2 @1.87 min |
| 16 | 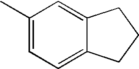 | 5-(2-Methylcarbamoyl-pyridin-4-yloxy)-2,3- dihydro-indole-1-carboxylic acid- indan-5-ylamide | 429.0 @2.22 min |
| 17 | 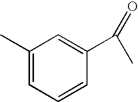 | 5-(2-Methylcarbamoyl-pyridin-4-yloxy)-2,3- dihydro-indole-1-carboxylic acid (3-acetyl-phenyl)-lamide | 431.2 @1.85 min |
| 18 | 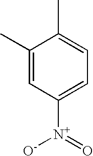 | 5-(2-Methylcarbamoyl-pyridin-4-yloxy)-2,3- dihydro-indole-1-carboxylic acid (2-methyl-5-nitro-phenyl)-lamide | 448.0 @2.07 min |
| 19 | 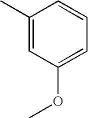 | 5-(2-Methylcarbamoyl-pyridin-4-yloxy)-2,3- dihydro-indole-1-carboxylic acid (3-methoxy-phenyl)-lamide | 419.2@ 1.97 min |
| 20 | 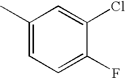 | 5-(2-Methylcarbamoyl-pyridin-4-yloxy)-2,3- dihydro-indole-1-carboxylic acid (3-chloro-4-fluoror-phenyl)-lamide | 441.0 @2.22 min |
| 21 | 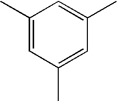 | 5-(2-Methylcarbamoyl-pyridin-4-yloxy)-2,3- dihydro-indole-1-carboxylic acid (3-,5-dimethyl-phenyl)-amide | 417.2 @2.12 min |
| 22 | 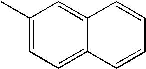 | 5-(2-Methylcarbamoyl-pyridin-4-yloxy)-2,3- dihydro-indole-1-carboxylic acid naphthalen-2-yl-amide | 439.2 @2.25 min |
| 23 | 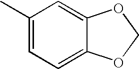 | 5-(2-Methylcarbamoyl-pyridin-4-yloxy)-2,3- dihydro-indole-1-carboxylic acid benzo[1,3]dioxol-5-ylamide | 433.0 @1.90 min |
| 24 | 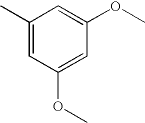 | 5-(2-Methylcarbamoyl-pyridin-4-yloxy)-2,3- dihydro-indole-1-carboxylic acid (3,5-dimethoxy-phenyl)-amide | 449.0 @2.02 min |
| 25 | 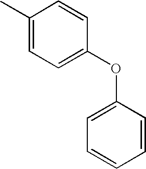 | 5-(2-Methylcarbamoyl-pyridin-4-yloxy)-2,3- dihydro-indole-1-carboxylic acid (4-phenoxy-phenyl)-amide | 481.0 @2.35 min |
| 26 | 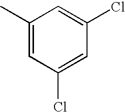 | 5-(2-Methylcarbamoyl-pyridin-4-yloxy)-2,3- dihydro-indole-1-carboxylic acid (3,5-dichloro-phenyl)-amide | 457.0 @2.47 min |
| 27 | 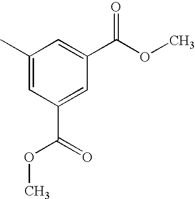 | 5-(2-Methylcarbamoyl-pyridin-4-yloxy)-2,3- dihydro-indole-1-carboxylic acid (3-acetyl-phenyl)-lamide | 505.2@ 2.17 min |
| 28 | 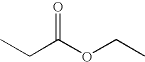 | ({1-[5-(2-Methylcarbamoyl-pyridin-4-yloxy)- 2,3-dihydro-indol-1-yl]-methanoyl}- amino)-acetic acid ethyl ester | 399.2 @1.55 min |
| 29 | 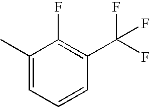 | 5-(2-Methylcarbamoyl-pyridin-4-yloxy)-2,3- dihydro-indole-1-carboxylic acid (2-fluoro-3- tirfluoromethyl-phenyl)-amide | 475.0 @2.25 min |
| 30 | 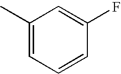 | 5-(2-Methylcarbamoyl-pyridin-4-yloxy)-2,3- dihydro-indole-1-carboxylic acid (3-fluoro- phenyl)-amide | 464.2 @2.29 min |
| 31 | 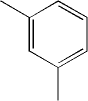 | 5-(2-Methylcarbamoyl-pyridin-4-yloxy)-2,3- dihydro-indole-1-carboxylic acid (3-methyl- phenyl)-amide | 403.2 @2.15 min |

### Examples 32-48

Following the procedures described above in Example 1(a) except substituting the appropriate amine for N-methyl amine the following title compounds were prepared. Thus, the following 4-Chloro-pyridine-2-carboxylic acid amides were prepared as described above in Example 1(a) starting from picolinic acid and corresponding amine:

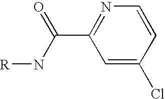

| Intermediate # | R | Compound Name | ¹H—NMR (CDCl₃) |
|---|---|---|---|
| 32a | CH₃CH₂— | 4-Chloro-pyridine-2- carboxylic acid ethylamide | 1.29 (t, 3H, J=7.6 Hz), 3.53 (q, 2H, J=7.6 Hz), 7.48 (q, 1H, J=2 Hz), 8.02 (br s, 1H) 8.23 (d, 1H, J=2 Hz) 8.45 (d, 1H, J=4.8 Hz) |
| 33a | CH₃CH₂CH₂— | 4-Chloro-pyridine-2- carboxylic acid propylamide | 1.00 (t, 3H, J=7.6 Hz), 1.66 (m, 2H), 3.44 (q, 2H, J=7.6 Hz), 7.43 (q, 1H, J=2 Hz), 8.02 (br s, 1H) 8.21 (d, 1H, J=2 Hz) 8.46 (d, 1H, J=4.8 Hz) |
| 34a | 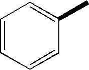 | 4-Chloro-pyridine-2- carboxylic acid phenylamide | 7.18-7.50 (m, 6H) 8.33 (m, 1H) 8.45 (s, 1H) 9.93 (br s, 1H) |
| 35a | 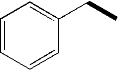 | 4-Chloro-pyridine-2- carboxylic acid benzylamide | 4.69 (d, 2H, J=6 Hz), 7.20-7.48 (m, 6H) 8.31 (m, 2H) 8.45 (s, 1H) |
| 36a | 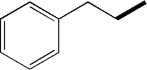 | 4-Chloro-pyridine-2- carboxylic acid phenethyl- amide | 2.87 (m, 2H), 3.05 (m, 2H), 7.18-7.44 (m, 6H) 8.06 (br s, 1H) 8.26 (s, 1H) 8.43 (s, 1H) |
| 37a | 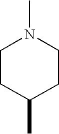 | 4-Chloro-pyridine-2- carboxylic acid (1-methyl- piperidin-4-yl)-amide | 1.64 (m, 2H), 2.02 (m, 2H), 2.18 (m, 2H), 2.32 (s, 3H) 2.85 (m, 2H) 3.95 (m, 1H) 7.44 (q, 1H, J=2 Hz), 7.95 (br s, 1H) 8.25 (d, 1H, J=2 Hz) 8.41 (d, 1H, J=4.8 Hz) |
| 38a | 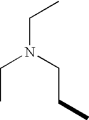 | 4-Chloro-pyridine-2- carboxylic acid (2- diethylamino-ethyl)-amide | 1.06 (m, 6H), 1.80 (m, 2H), 1.93 (m, 2H), 2.59 (m, 6H) 3.88 (m, 2H) 7.41 (q, 1H, J=2 Hz), 8.21 (d, 1H, J=2 Hz) 8.45 (d, 1H, J=4.8 Hz) 8.97 (br s, 1H) |
| 39a | 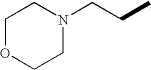 | 4-Chloro-pyridine-2- carboxylic acid (2-morpholin- 4-yl-ethyl)-amide | 2.56 (m, 4H), 2.64 (m, 2H), 3.62 (m, 2H), 3.77 (m, 4H) 7.44 (q, 1H, J=2 Hz), 8.21 (d, 1H, J=2 Hz) 8.32 (br s, 1H) 8.49 (d, 1H, J=4.8 Hz) |
| 40a | 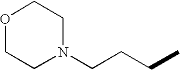 | 4-Chloro-pyridine-2- carboxylic acid (3-morpholin- 4-yl-propyl)-amide | 1.45 (m, 4H), 1.88 (m, 2H), 2.60 (m, 4H), 3.63 (m, 2H) 3.86 (m, 4H) 7.45 (q, 1H, J=2 Hz), 8.22 (d, 1H, J=2 Hz) 8.58 (d, 1H, J=4.8 Hz) 8.98 (br s, 1H) |
| 41a | 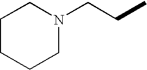 | 4-Chloro-pyridine-2- carboxylic acid (2-piperidin-1- yl-ethyl)-amide | 1.44 (m, 2H), 1.64 (m, 2H), 2.46-2.68 (m, 6H), 3.82 (m, 2H) 3.86 (m, 4H) 7.43 (q, 1H, J=2 Hz), 8.33 (d, 1H, J=2 Hz) 8.41 (br s, 1H) 8.47 (d, 1H, J=4.8 Hz) |
| 42a | 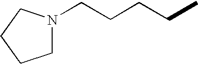 | 4-Chloro-pyridine-2- carboxylic acid (4-pyrrolidin- 1-yl-butyl)-amide | 1.65 (m, 4H), 1.83 (m, 4H), 2.03 (m, 2H), 2.54 (m, 2H) 3.76 (m, 4H) 7.43 (q, 1H, J=2 Hz), 8.21 (m, 2H) 8.45 (d, 1H, J=4.8 Hz) |
| 43a | 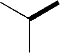 | 4-Chloro-pyridine-2- carboxylic acid isopropylamide | 1.31 (d, 6H, J=6 Hz), 4.29 (m, 1H), 7.43 (q, 1H, J=2 Hz) 7.69 (br s, 1H) 8.22 (d, 1H, J=2 Hz) 8.46 (d, 1H, J=4.8 Hz) |
| 44a | 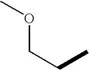 | 4-Chloro-pyridine-2- carboxylic acid (2-methoxy- ethyl)-amide | 3.42 (s, 3H), 3.66 (m, 2H), 3.70 (m, 2H) 7.44 (q, 1H, J=2 Hz) 8.22 (br s, 2H) 8.48 (d, 1H, J=4.8 Hz) |
| 45a | 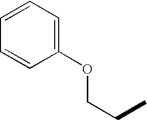 | 4-Chloro-pyridine-2- carboxylic acid (2-phenoxy- ethyl)-amide | 4.00 (m, 2H), 4.19 (m, 2H) 6.98 (m, 4H) 7.31 (m 2H) 8.23 (d, 1H, J=2 Hz) 8.41 (br s, 1H) 8.48 (d, 1H, J=4.8 Hz) |
| 46a | 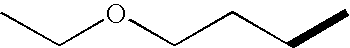 | 4-Chloro-pyridine-2- carboxylic acid (3-ethoxy- propyl)-amide | 1.26 (t, 3H, J=7.2 Hz) 1.92 (m, 3H), 3.56 (m, 6H), 7.44 (q, 1H, J=2 Hz) 8.21 (d, 1H, J=2 Hz) 8.46 (m, 2H) |
| 47a | 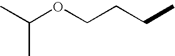 | 4-Chloro-pyridine-2- carboxylic acid (3-isopropoxy- propyl)-amide | 1.22 (m, 8H) 1.92 (m, 2H), 3.62 (m, 5H), 7.43 (q, 1H, J=2 Hz) 8.21 (d, 1H, J=2 Hz) 8.46 (d, 1H, J=4.8 Hz) 8.84 (m, 2H) |
| 48a | 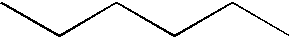 | 4-Chloro-pyridine-2- carboxylic acid hexylamide | 0.93 (m, 3H) 1.45 (m, 6H), 1.66 (m, 2H), 3.50 (m, 2H), 7.44 (q, 1H, J=2 Hz) 7.99 (m, 2H) 8.23 (d, 1H, J=2 Hz) 8.46 (m, 2H) |

Then, the following 4-(1-Acetyl-2,3-dihydro-1H-indol-5-yloxy)-pyridine-2-carboxylic acid amides were prepared as described above in Example 1(b) starting from 1-acetyl-2,3-dihydro-5-hydroxyindole and corresponding 4-Chloro-pyridine-2-carboxylic acid amide:

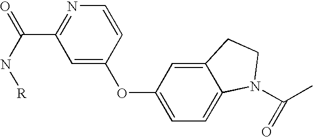

| | | | LC Mass Spectra |
|---|---|---|---|
| Example # | R | Compound Name | [M + H] @ ret time |
| 32 | CH₃CH₂— | 4-(1-Acetyl-2,3-dihydro-1H-indol- 5-yloxy)-pyridine-2-carboxylic acid ethylamide | 326.4 @ 1.66 min |
| 33 | CH₃CH₂CH₂— | 4-(1-Acetyl-2,3-dihydro-1H-indol- 5-yloxy)-pyridine-2-carboxylic acid propylamide | 340.2 @ 1.77 min |
| 34 | 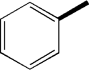 | 4-(1-Acetyl-2,3-dihydro-1H-indol- 5-yloxy)-pyridine-2-carboxylic acid phenylamide | 352.2 @ 1.83 min |
| 35 | 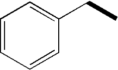 | 4-(1-Acetyl-2,3-dihydro-1H-indol- 5-yloxy)-pyridine-2-carboxylic acid benzylamide | 374.2 @ 2.17 min |
| 36 | 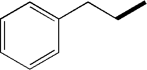 | 4-(1-Acetyl-2,3-dihydro-1H-indol- 5-yloxy)-pyridine-2-carboxylic acid phenethyl-amide | 388.2 @ 1.97 min |
| 37 | 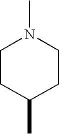 | 4-(1-Acetyl-2,3-dihydro-1H-indol- 5-yloxy)-pyridine-2-carboxylic acid (1-methyl-piperidin-4-yl)-amide | 402.2 @ 2.05 min |
| 38 | 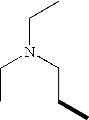 | 4-(1-Acetyl-2,3-dihydro-1H-indol- 5-yloxy)-pyridine-2-carboxylic acid (2-diethylamino-ethyl)-amide | 395.2 @ 1.28 min |
| 39 | 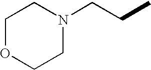 | 4-(1-Acetyl-2,3-dihydro-1H-indol- 5-yloxy)-pyridine-2-carboxylic acid (2-morpholin-4-yl-ethyl)-amide | 411.4 @ 1.30 min |
| 40 | 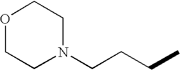 | 4-(1-Acetyl-2,3-dihydro-1H-indol- 5-yloxy)-pyridine-2-carboxylic acid (3-morpholin-4-yl-propyl)-amide | 425.0 @ 1.37 min |
| 41 | 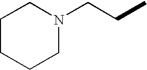 | 4-(1-Acetyl-2,3-dihydro-1H-indol- 5-yloxy)-pyridine-2-carboxylic acid (2-piperidin-1-yl-ethyl)-amide | 409.2 @ 1.30 min |
| 42 | 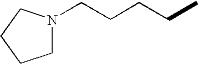 | 4-(1-Acetyl-2,3-dihydro-1H-indol- 5-yloxy)-pyridine-2-carboxylic acid (2-pyrrolidin-1-yl-butyl)-amide | 423.0 @ 1.35 min |
| 43 | 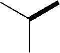 | 4-(1-Acetyl-2,3-dihydro-1H-indol- 5-yloxy)-pyridine-2-carboxylic acid isopropylamide | 340.0 @ 2.90 min |
| 44 | 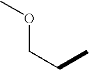 | 4-(1-Acetyl-2,3-dihydro-1H-indol- 5-yloxy)-pyridine-2-carboxylic acid (2-methoxy-ethyl)-amide | 356.0 @ 2.82 min |
| 45 | 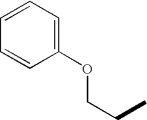 | 4-(1-Acetyl-2,3-dihydro-1H-indol- 5-yloxy)-pyridine-2-carboxylic acid (2-phenoxy-ethyl)-amide | 418.4 @ 3.05 min |
| 46 | 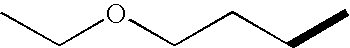 | 4-(1-Acetyl-2,3-dihydro-1H-indol- 5-yloxy)-pyridine-2-carboxylic acid (3-ethoxy-propyl)-amide | 384.2 @ 1.89 min |
| 47 | 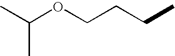 | 4-(1-Acetyl-2,3-dihydro-1H-indol- 5-yloxy)-pyridine-2-carboxylic acid (3-isopropoxy-propyl)-amide | 398.2 @ 2.05 min |
| 48 | 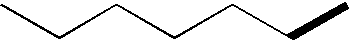 | 4-(1-Acetyl-2,3-dihydro-1H-indol- 5-yloxy)-pyridine-2-carboxylic acid hexylamide | 382.4 @ 2.42 min |

### Examples 49-65

Following the procedures described above in Example 1(a), 1(b), and 1(c), except substituting the appropriate amine for N-methyl amine and using the procedure below for the coupling using 3-trifluoromethylphenylisocyanate, the following title compounds 5-(2-substitutedcarbamoyl-pyridin-4-yloxy)-2,3-dihydro-indole-1-carboxylic acid (3-trifluoromethyl-phenyl)-amides were prepared.

Hydrolysis of the N-acetyl compounds was executed as described above in Example 1(c) starting from corresponding 4-(1-Acetyl-2,3-dihydro-1H-indol-5-yloxy)-pyridine-2-carboxylic acid amides to give the title compounds, which were used in the next step without further purification. The coupling was performed as follows: To a solution of 4-(2,3-dihydro-1H-indol-5-yloxy)-pyridine-2-carboxylic acid amide (0.2 mmol) in NMP (0.5 mL) was added 3-trifiuoromethyl-phenylisocyanate (0.3 mmol). The reaction was stirred at rt for for 30 min and then purified by preparative HPLC. Pure desired product was then isolated as its trifluoroacetic salt.

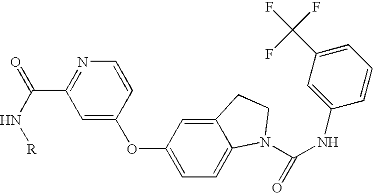

| | | | LC Mass |
|---|---|---|---|
| | | | Spectra |
| | | | [M + H] |
| Example # | R | Compound Name | @ ret time |
| 49 | CH₃CH₂— | 5-(2-Ethylcarbamoyl-pyridin-4-yloxy)-2,3- dihydro-indole-1-carboxylic acid (3- trifluoromethyl-phenyl)-amide | 471.2 @2.25 min |
| 50 | CH₃CH₂CH₂— | 5-(2-Propylcarbamoyl-pyridin-4-yloxy)- 2,3-dihydro-indole-1-carboxylic acid (3- trifluoromethyl-phenyl)-amide | 485.2 @2.25 min |
| 51 | 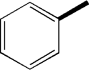 | 5-(2-Phenylcarbamoyl-pyridin-4-yloxy)- 2,3-dihydro-indole-1-carboxylic acid (3- trifluoromethyl-phenyl)-amide | 519.2 @2.62 min |
| 52 | 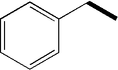 | 5-(2-Benzylcarbamoyl-pyridin-4-yloxy- 2,3-dihydro-indole-1-carboxylic acid (3- trifluoromethyl-phenyl)-amide | 533.2 @2.49 min |
| 53 | 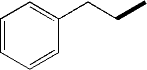 | 5-(2-Phenethylcarbamoyl-pyridin-4- yloxy)-2,3-dihydro-indole-1-carboxylic acid (3-trifluoromethyl-phenyl)-amide | 547.2 @2.54 min |
| 54 | 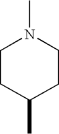 | 5-[2-(1-Methyl-piperidin-4-ylcarbamoyl)- pyridin-4-yloxy]-2,3-dihydro-indole-1- carboxylic acid (3- trifluoromethyl-phenyl)-amide | 540.2 @1.95 min |
| 55 | 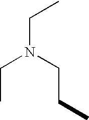 | 5-[2-(3-Diethylamino-propylcarbamoyl)- pyridin-4-yloxy]-2,3-dihydro-indole-1- carboxylic acid (3-trifluoromethyl-phenyl)- amide | 556.2 @2.04 min |
| 56 | 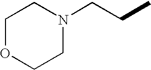 | 5-[2-(2-Morpholin-4-yl-ethylcarbamoyl)- pyridin-4-yloxy]-2,3-dihydro-indole-1- carboxylic acid (3-trifluoromethyl-phenyl)- amide | 556.0 @1.94 min |
| 57 | 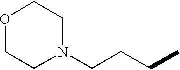 | 5-[2-(3-Morpholin-4-yl-propylcarbamoyl)- pyridin-4-yloxy]-2,3-dihydro-indole-1- carboxylic acid (3-trifluoromethyl-phenyl)- amide | 570.0 @1.94 min |
| 58 | 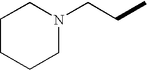 | 5-[2-(2-Piperidin-1-yl-ethylcarbamoyl)- pyridin-4-yloxy]-2,3-dihydro-indole-1- carboxylic acid (3-trifluoromethyl-phenyl)- amide | 554.4 @2.09 min |
| 59 | 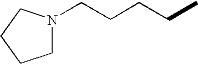 | 5-[2-(4-Pyrrolidin-1-yl-butylcarbamoyl)- pyridin-4-yloxy]-2,3-dihydro-indole-1- carboxylic acid (3-trifluoromethyl-phenyl)- amide | 568.2 @2.22 min |
| 60 | 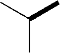 | 5-(2-Isopropylcarbamoyl-pyridin-4-yloxy)- 2,3-dihydro-indole-1-carboxylic acid (3-trifluoromethyl-phenyl)-amide | 484.6 @2.50 min |
| 61 | 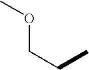 | 5-[2-(2-Methoxy-ethylcarbamoyl)-pyridin- 4-yloxy]-2,3-dihydro-indole-1-carboxylic acid (3-trifluoromethyl-phenyl)-amide | 501.2 @2.35 min |
| 62 | 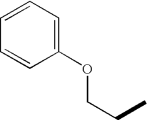 | 5-[2-(2-Phenoxy-ethylcarbamoyl)-pyridin- 4-yloxy]-2,3-dihydro-indole-1-carboxylic acid (3-trifluoromethyl-phenyl)-amide | 563.0 @2.71 min |
| 63 | 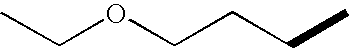 | 5-[2-(3-Ethoxy-propylcarbamoyl)-pyridin- 4-yloxy]-2,3-dihydro-indole-1-carboxylic acid (3-trifluoromethyl-phenyl)-amide | 529.2 @2.55 min |
| 64 | 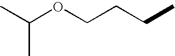 | 5-[2-(3-Isopropoxy-propylcarbamoyl)- pyridin-4-yloxy]-2,3-dihydro-indole-1- carboxylic acid (3-trifluoromethyl-phenyl)- amide | 543.4 @2.62 min |
| 65 | 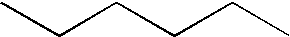 | 5-(2-Hexylcarbamoyl-pyridin-4-yloxy)- 2,3-dihydro-indole-1-carboxylic acid (3- trifluoromethyl-phenyl)-amide | 527.2 @2.87 min |

### Example 66

### 5-(2-Methylcarbamoyl-pyridin-4-yloxy)-2,3-dihydro-indole-1-carboxylic acid [4-(4-methyl-piperazin-1-ylmethyl)-phenyl]-amide

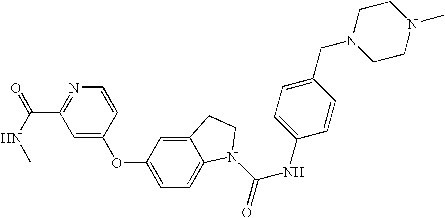

a) 1-Methyl-4-(4-nitro-benzyl)-piperazine. 4-Nitrobenzyl chloride (0.5 g, 2.91 mmol) was dissolved in acetone (10 mL), and to this solution was added K₂CO₃ (0.8 g, 5.8 mmol) and 1-methylpiperazine (0.39 mL, 3.5 mmol). After heating at reflux for one hour, the mixture was cooled, the excess K₂CO₃ removed by filtration, and the acetone evaporated in vacuo. Column chromatography afforded 1-methyl-4-(4-nitro-benzyl)-piperazine as an off-white solid (0.59 g, 86% yield): MS (M+1) 236; ¹H NMR (400 MHz, CDCl₃) δ8.17 (d, J=8.8 Hz, 2H), 7.50 (d, J=8.8 Hz, 2H), 3.66 (s, 2H), 2.75 (bs, 4H), 2.55 (bs, 4H), 2.42 (s, 3H).

b) 4-(4-Methyl-piperazin-1-ylmethyl)-phenylamine. 1-Methyl-4-(4-nitro-benzyl)-piperazine (100 mg, 0.43 mmol) was added to a flask containing 5 mg Pt/C (5% w/w, 62% H₂O content) in EtOH (2.1 mL). The reaction mixture was stirred under an atmosphere of H₂ until the reaction was complete by LCMS analysis (4h). The mixture was filtered, rinsed with EtOH, and concentrated to afford 4-(4-methyl-piperazin-1-ylmethyl)-phenylarnine as a yellow oil, which was sufficiently pure for the next step (97 mg, 100%): MS (M+1) 206; ¹H NMR (400 MHz, CDCl₃) δ7.10 (d, J=8.4 Hz, 2H), 6.63 (d, J=8.4 Hz, 2H), 3.67 (bs, 2H), 3.49 (s, 2H), 2.66 (bs, 8H), 2.42 (s, 3).

c) 5-(2-Methylcarbamoyl-pyridin-4-yloxy)-2,3-dihydro-indole-1-carboxylic acid [4-(4-methyl-piperazin-1-ylmethyl)-phenyl]-amide. To a 25 mL flask was added under a nitrogen atmosphere phenyl chloroformate (0.0245 mL, 0.195 mmol) and THF (0.65 mL). The mixture was cooled to 0° C. and 4-(4-methyl-piperazin-1-ylmethyl)-phenylamine (0.04 g, 0.195 mmol) in THF (0.6 mL) was added dropwise via syringe over 30 min. Et₃N (0.028 mL, 0.197 mmol) was added, and the heterogeneous mixture is allowed to warm slowly to room temperature over two hours. At this time, the mixture of the crude carbamate was transferred via cannula to a flask containing the pyridyl indoline (0.052 g, 0.195 mmol) and THF (0.4 mL). Additional Et₃N (0.085 mL, 0.605 mmol) was added, the septum replaced with a condenser, and the whole mixture was refluxed under nitrogen overnight. Upon cooling, the mixture was treated with H₂O, CH₂Cl₂, and sat. aq. NaHCO₃. The layers were separated, and the aqueous portion extracted further with CH₂Cl₂ (2×). Purification on reverse-phase HPLC, followed by treatment with aq. NaHCO₃ and extraction with CH₂Cl₂ provided 5-(2-methylcarbamoyl-pyridin-4-yloxy)-2,3-dihydro-indole-1-carboxylic acid [4-(4-methyl-piperazin-1-ylmethyl)-phenyl]-amide as the free-base (8.8 mg, 10% yield): MS (M+1) 501; ¹H NMR (400 MHz, CDCl₃) δ8.36 (d, J=5.6 Hz, 1H), 7.98 (m, 2H), 7.67 (d, J=2.4 Hz, 1H), 7.40 (d, J=8.0 Hz, 2), 7.29 (d, J=7.6 Hz, 2H), 6.95 (m, 3H), 6.44 (s, 1H), 4.16 (t, J=8.0 Hz, 2H), 3.48 (s, 2H), 3.26 (t, J=7.6 Hz, 2H), 3.01 (d, J=5.2 Hz, 3H), 2.49 (m, 8H), 2.29 (s, 3H).

### Example 67

### 5-(2-Methylcarbamoyl-pyridin-4-yloxy)-2,3-dihydro-indole-1-carboxylic acid [3-(4-methyl-piperazin-1-ylmethyl)-phenyl]-amide:

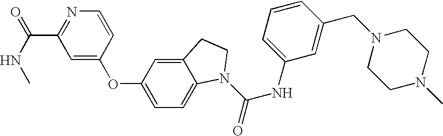

Following the procedure of Example 66(a)-66(c), except substituting 3-nitrobenzyl bromide for 4-nitrobenzyl chloride in Example 66(a), the following compounds were prepared:

a) 1-Methyl-4-(3-nitro-benzyl)-piperazine: 67% yield; MS (M+1) 236; ¹H NMR (400 MHz, CDCl₃) 38.21 (s, 1H), 8.11 (ddd, J=0.8, 1.2, 8.4 Hz, 1H), 7.64 (d, J=7.2 Hz, 1H), 7.48 (t, J=8.0 Hz, 1H), 3.61 (s, 2H), 2.59 (bs, 8H), 2.39 (s, 3H).

b) 3-(4-Methyl-piperazin-1-ylmethyl)-phenyl-amine: 83% yield; MS (M+1) 206; ¹H NMR (400 MHz, CDCl₃) δ7.06 (t, J=8.0 Hz, 1H), 6.5-6.7 (m, 3H), 3.63 (bs, 2H), (s, 2H), 2.47 (bs, 8H), 2.29 (s, 3H).

c) 5-(2-Methylcarbamoyl-pyridin-4-yloxy)-2,3-dihydro-indole-1-carboxylic acid [3-(4-methyl-piperazin-1-ylmethyl)-phenyl]-amide: 35% yield; ¹H NMR (400 MHz, CDCl₃) δ8.36 (d, J=5.6 Hz, 1H), 7.98 (m, 2H), 7.67 (d, J=2.8 Hz, 1H), 7.45 (dd, J=2.1, 8.0 Hz, 1H), 7.35 (s, 1H), 7.29 (d, J=7.6 Hz, 1H), 7.05 (d, J=7.6 Hz, 1H), 6.95 (m, 3H), 6.48 (s, 1H), 4.14 (t, J=8.8 Hz, 2H), 3.50 (s, 2H), 3.26 (t, J=8.4 Hz, 2H), 3.01 (d, J=5.2 Hz, 3H), 2.50 (bs, 8H), 2.30 (s, 3H).

### Example 68

### 5-(2-Methylcarbamoyl-pyridin-4-yloxy)-2,3-dihydro-indole-1-carboxylic acid [4-(4-methyl-piperazin-1-ylmethyl)-3-trifluoromethyl-phenyl]-amide

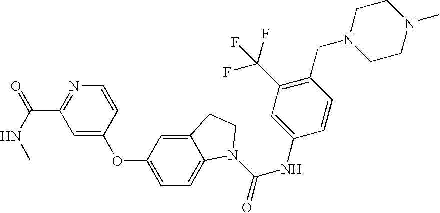

a) 1-Methyl-4-(nitro-trifluoromethyl-benzyl)-piperazine: To a suspension of 4-nitro-2-(trifluoromethyl)toluene (0.5 g, 2.44 mmol) in AcOH (1.9 mL) was added NBS (0.651 g, 3.66 mmol) and benzoyl peroxide (6 mg, 0.024 mmol), and mixture was heated at reflux overnight. Upon cooling, the solvent was removed in vacuo, EtOAc and aq. NaHCO₃ were added, and the layers were separated. The organic layer was dried (Na₂SO₄), filtered, and concentrated to afford crude benzyl bromide (700 mg), which was used in the next reaction without further purification.

To a solution of crude 1-bromomethyl-4-nitro-2-trifluoromethyl-benzene (400 mg, 1.41 mmol) in CH₂Cl₂ (2.8 mL) was added Et₃N (0.197 mL, 1.41 mmol) and 1-methylpiperazine (0.157 mL, 1.41 mmol). After stirring for two hours, aq. NaHCO₃ was added, and the mixture was extracted with CH₂Cl₂ (2×). The combined organic layers were dried over Na₂SO₄, filtered, concentrated and the resulting residue purified by silica gel chromatography to afford 1-methyl-4-(nitro-trifluoromethyl-benzyl)-piperazine (249 mg, 58% yield): MS (M+1) 304; ¹H NMR (400 MHz, CDCl₃) δ8.51 (d, J=2.0 Hz, 1H), 8.37 (dd, J=2.0, 8.4 Hz, 1H), 8.10 (d, J=8.4 Hz, 1H), 3.75 (s, 2H), 2.49 (m, 8H), 2.32 (s, 3H).

Following the procedure of Example 66(b) and 66(c), except substituting 1-methyl-4-(nitro-triluoromethyl-benzyl)-piperazine for 1-methyl-4-(4-nitro-benzyl)-piperazine in Example 66(a), the following compounds were prepared:

b) 4-(4-Methyl-piperazin-1-ylmethyl)-3-trifluoromethyl-phenyl-amine: 70% yield; MS (M+1) 274; ¹H NMR (400 MHz, CDCl₃) δ7.47 (d, J=8.4 Hz, 1H), 6.91 (d, J=2.4 Hz, 1H), 6.79 (dd, J=2.4, 8.4 Hz, 1H), 3.75 (bs, 2H), 3.53 (s, 2H), 2.48 (bs, 8H), 2.29 (s, 3H).

c) 5-(2-Methylcarbamoyl-pyridin-4-yloxy)-2,3-dihydro-indole-1-carboxylic acid [4-(4-methyl-piperazin-1-ylmethyl)-3-trifluoromethyl-phenyl]-amide: 16% yield; MS (M+1) 569; ¹H NMR (400 MHz, CDCl₃) δ8.36 (d, J=5.2 Hz, 1H), 8.00 (m, 2H), 7.69 (m, 4H), 6.95 (m, 3H), 6.56 (s, 1H), 4.16 (t, J=8.4 Hz, 2H), 3.62 (s, 2H), 3.28 (t, J=8.4 Hz, 2H), 3.01 (d, J=5.2 Hz, 3H), 2.50 (m, 8H), 2.31 (s, 3H).

### Example 69

Following the procedure of Example 68(a)-68(c), except substituting morpholine for 1-methylpiperazine in Example 66(a), the following compound was prepared:

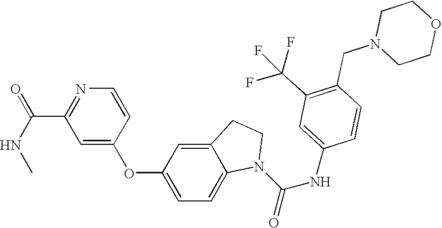

a) 4-(Nitro-trifluoromethyl-benzyl)-morpholine. 59% yield; MS (M+1) 291; ¹H NMR (400 MHz, CDCl₃) δ8.51 (d, J=2.4 Hz, 1H), 8.38 (dd, J=2.4, 8.4 Hz, 1H), 8.12 (d, J=8.4 Hz, 1H), 3.75 (dd, J=4.4, 4.8 Hz, 6H), 2.51 (dd, J=4.4, 4.8 Hz, 4H).

b) 4-Morpholin-4-ylmethyl-3-trifluoromethyl-phenyl-amine: 84% yield; MS (M+1) 261; ¹H NMR (400 MHz, CDCl₃) δ7.51 (d, J=7.2 Hz, 1H), 6.92 (d, J=2.4 Hz, 1H), 6.80 (dd, J=2.0, 8.4 Hz, 1H), 3.78 (bs, 2H), 3.71 (dd, J=4.4, 4.8 Hz, 4H), 2.44 (dd, J=4.4, 4.8 Hz, 4H).

c) 5-2-Methylcarbamoyl-pyridin-4-yloxy)-2,3-dihydro-indole-1-carboxylic acid (4-morpholin-4-ylmethyl-3-tritluoromethyl-phenyl)-amide: 28% yield; MS (M+1) 556; ¹H NMR (400 MHz, CDCl₃) δ8.37 (d, J=5.6 Hz, 1H), 8.01 (m, 2H), 7.69 (m, 4H), 6.96 (m, 3H), 6.58 (s, 1H), 4.16 (t, J=8.4 Hz, 2H), 3.72 (dd, J=4.4, 4.8 Hz, 4H), 3.62 (s, 2H), 3.28 (d, J=8.4 Hz, 2H), 3.01 (d, 5.2 Hz, 3H), 2.47 (dd, J=4.4, 4.8 Hz, 4H).

### Examples 70-82

4-(2,3-Dihydro-1-H-indol-6-yloxy)-pyridine-2-carboxylic acid methylamide (Example 1(c), 27 mg, 0.1 mmol) and 1,1′carbonyl-diimidazole (32 mg, 0.2 mmol) were dissolved in DMF 1.0 ml) and stirred at rt for 5 min followed by addition of the corresponding substituted acetic acid (0.2 mmol) and triethylamine (19 ul, 0.1 mmol). This mixture was stirred at rt overnight. The crude reaction mixture was chromatographed on reverse phase column eluting 10%-90% acetonitrile/0.1% TFA water to afford the following title compounds after removal:

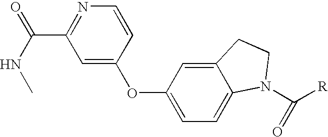

| | | | LC Mass |
|---|---|---|---|
| | | | Spectra |
| | | | [M + H] @ |
| Example # | R | Compound Name | ret time |
| 70 | 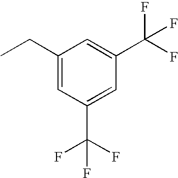 | 4-{1-[2-(Bis-trifluoromethyl-phenyl)-ethanoyl]- 2,3-dihydro-1H-indol-5-yloxy}-pyridine-2- carboxylicacid methylamide | 542 @2.88 min |
| 71 | 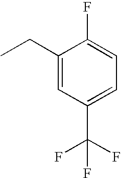 | 4-{1-[2-(Fluoro-trifluoromethyl-phenyl)- ethanoyl]-2,3-dihydro-1H-indol-5-yloxy}- pyridine-2-carboxylic acid methylamide | 474 @2.34 min |
| 72 | 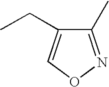 | 4-{1-[2-(3-Methyl-isoxazol-4-yl)-ethanoyl]-2,3- dihydro-1H-indol-5-yloxy}-pyridine-2- carboxylic acid methylamide | 393 @1.95 min |
| 73 | 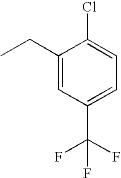 | 4-{1-[2-(Chloro-trifluoromethyl-phenyl)- ethanoyl]-2,3-dihydro-1H-indol-5-yloxy}- pyridine-2-carboxylic acid methylamide | 490 @2.32 min |
| 74 | 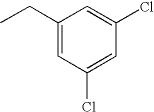 | 4-{1-[2-(3,5-Dichloro-phenyl)-ethanoyl]-2,3- dihydro-1H-indol-5-yloxy}-pyridine-2- carboxylic acid methylamide | 456 @2.21 min |
| 75 | 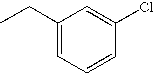 | 4-{1-[2-(Fluoro-trifluoromethyl-phenyl)- ethanoyl]-2,3-dihydro-1Hindol-5-yloxy}- pyridine-2-carboxylic acid methylamide | 422 @1.98 min |
| 76 | 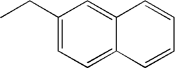 | 4-[1-(2-Naphthalen-2-yl-ethanoyl)-2,3-dihydro- 1H-indol-5-yloxy]-pyridine-2-carboxylic acid methylamide | 438 @2.67 min |
| 77 | 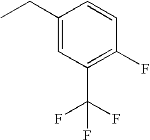 | 4-{1-[2-(Fluoro-trifluoromethyl-phenyl)- ethanoyl]-2,3-dihydro-1Hindol-5-yloxy}- pyridine-2-carboxylic acid methylamide | 474 @2.40 min |
| 78 | 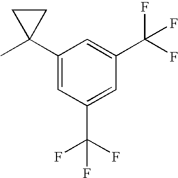 | 4-(1-{1-[1-(Bis-trifluoromethyl-phenyl)- cyclopropyl]-methanoyl}-2,3-dihydro-1H-indol- 5-yloxy)-pyridine-2-carboxylic acid methylamide | 540 @1.85 min |
| 79 | 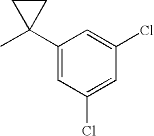 | 4-(1-{1-[1-(3,4-Dichloro-phenyl)-cyclopropyl]- methanoyl}-2,3-dihydro-1H-indol-5-yloxy)- pyridine-2-carboxylic acid methylamide | 482 @2.55 min |
| 80 | 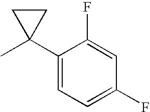 | 4-(1-{1-[1-(2,4-Difluoro-phenyl)-cyclopropyl]- methanoyl}-2,3-dihydro-1H-indol-5-yloxy)- pyridine-2-carboxylic acid methylamide | 450 @1.65 min |
| 81 | 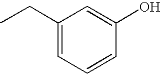 | 4-{1-[2-(3-Hydroxy-phenyl)-ethanoyl]-2,3- dihydro-1H-indol-5-yloxy}-pyridine-2- carboxylic acid methylamide | 404 @1.89 min |
| 82 | 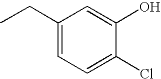 | 4-{1-[2-(4-Chloro-3-hydroxy-phenyl)- ethanoyl]-2,3-dihydro-1H-indol-5-yloxy}- pyridine-2-carboxylic acid methylamide | 438 @1.95 min |

### Examples 83-86

4-{1-[2-3-Hydroxy-phenyl)-ethanoyl]-2,3-dihydro-1H-indol-5-yloxy)-pyridine-2-carboxylic acid methylamide (20 mg, 0.05 mmol) and the corresponding alkyl chloride HCl salt (0.075 mmol) were dissolved in 1.0 ml DMF and treated with potassium t-butoxide (12 mg, 0.1 mmol and potassium carbonate (14 mg, 0.1 mmol). The reaction mixture was stirred at rt overnight. The crude reaction mixture was chromatographed on reverse phase column eluting 10%-70% acetonitrile/0.1 % TFA water resulting in the following title compounds:

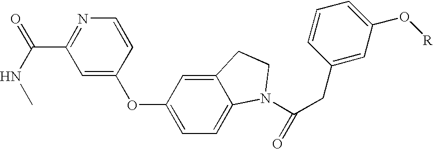

| | | | LC Mass |
|---|---|---|---|
| | | | Spectra |
| | | | [M + H] @ |
| Example # | R | Compound Name | ret time |
| 83 | 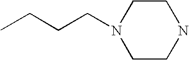 | 4-(1-{2-[3-(3-Piperazin-1-yl-propoxy)- phenyl]-ethanoyl}-2,3-dihydro-1H-indol-5- yloxy)-pyridine-2-carboxylic acid methylamide | 530 @2.25 min |
| 84 | 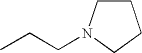 | 4-(1-{2-[3-(2-Pyrrolidin-1-yl-ethoxy)- phenyl]-ethanoyl}-2,3-dihydro-1H-indol-5- yloxy)-pyridine-2-carboxylic acid methylamide | 501 @1.83 min |
| 85 | 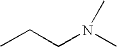 | 4-(1-{2-[3-(2-Dimethylamino-ethoxy)- phenyl]-ethanoyl}-2,3-dihydro-1H-indol-5- yloxy)-pyridine-2-carboxylic acid methylamide | 475 @2.12 min |
| 86 | 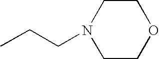 | 4-(1-{2-[3-(2-Morpholin-4-yl-ethoxy)- phenyl]-ethanoyl}-2,3-dihydro-1H-indol-5- yloxy)-pyridine-2-carboxylic acid methylamide | 518 @2.08 min |

### Examples 87-89

A mixture of 4-{1-[2-(4-Chloro-3-hydroxy-phenyl)-ethanoyl]-2,3-dihydro-1H-indol-5-yloxy}-pyridine-2-carboxylic acid methylamide (21 mg, 0.043 mmol), the corresponding alkyl chloride HCl salt (1.5 eq.), Et₃N (10 uL) and 18-crown-6 (1 mg) in acetonitrile (1 mL) with saturated K₂CO₃ solution (0.25 mL) were heated in a microwave at 180° or 15 min. The crude reaction mixture was chromatographed on reverse phase column eluting 10%-70% acetonitrile/0.1% TFA water resulting in the following title compounds:

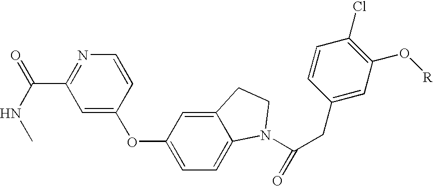

| | | | LC Mass |
|---|---|---|---|
| | | | Spectra |
| | | | [M + H] @ |
| Example # | R | Compound Name | ret time |
| 87 | 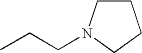 | 4-(1-{2-[3-Chloro-(2-pyrrolidin-1-yl- ethoxy)-phenyl]-ethanoyl}-2,3-dihydro- 1H-indol-5-yloxy)-pyridine-2-carboxylic acid methylamide | 535 @1.75 min |
| 88 | 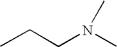 | 4-(1-{2-[3 Chloro-(2-dimethylamino- ethoxy)-phenyl]-ethanoyl}-2,3-dihydro- 1H-indol-5-yloxy)-pyridine-2-carboxylic acid methylamide | 509 @1.65 min |
| 89 | 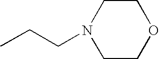 | 4-(1-{2-[3-Chloro-(2-morpholin-4-yl- ethoxy)-phenyl]-ethanoyl}-2,3-dihydro- 1H-indol-5-yloxy)-pyridine-2-carboxylic acid methylamide | 551 @1.66 min |

### Examples 90-99

4-(2,3-Dihydro-1-H-indol-6-yloxy)-pyridine-2-carboxylic acid methylamide (27 mg; 0.1 mmol) and Aryl-2-sulfonyl chloride (0.12 mmol) were dissolved in CH₂Cl₂ (1.0 ml). To this mixture, triethylamine (50 uL) was added at 0° C. and warmed up to rt. This mixture was stirred at rt overnight. The crude reaction mixture was chromatograph on reverse phase column by eluting with 10%-90% acetonitrile/0.1% TFA water resulting in the following title compounds:

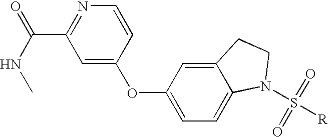

| | | | LC Mass |
|---|---|---|---|
| | | | Spectra |
| | | | [M + H] @ |
| Example # | R | Compound Name | ret time |
| 90 | 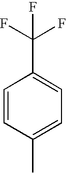 | 4-[1-(Methyl-trifluoromethyl- benzenesulfonyl)-2,3-dihydro-1H- indol-5-yloxy]-pyridine-2-carboxylic acid methylamide | 492 @2.34 min |
| 91 | 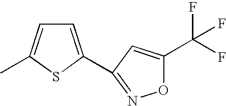 | 4-{1-[5-(5-Trifluoromethyl-isoxazol- 3-yl)-thiophene-2-sulfonyl]-2,3- dihydro-1H-indol-5-yloxy}-pyridine- 2-carboxylic acid methylamide | 551 @2.62 min |
| 92 | 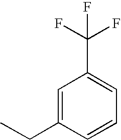 | 4-[1-(3-Trifluoromethyl- phenylmethanesulfonyl)-2,3-dihydro- 1H-indol-5-yloxy]-pyridine-2- carboxylic acid methylamide | 492 @2.61 min |
| 93 | 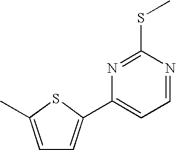 | 4-{1-[5-(2-Methylsulfanyl-pyrimidin- 4-yl)-thiophene-2-sulfonyl]-2,3- dihydro-1H-indol-5-yloxy}-pyridine- 2-carboxylic acid methylamide | 540.2 @2.77 min |
| 94 | 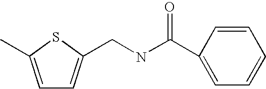 | 4-{1-[5-({[1-(4-Chloro-phenyl)- methanoyl]-amino}-methyl)- thiophene-2-sulfonyl]-2,3-dihydro- 1H-indol-5-yloxy}-pyridine-2- carboxylic acid methylamide | 583 @2.34 min |
| 95 | 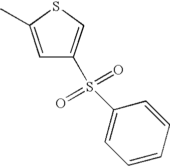 | 4-[1-(4-Benzenesulfonyl-thiophene-2- sulfonyl)-2,3-dihydro-1H-indol-5- yloxy]-pyridine-2-carboxylic acid methylamide | 566 @2.28 min |
| 96 | 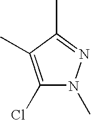 | 4-[1-(5-Chloro-1,3-dimethyl-1H- pyrazole-4-sulfonyl)-2,3-dihydro-1H- -indol-5-yloxy]-pyridine-2-carboxylic acid methylamide | 462 @2.05 min |
| 97 | 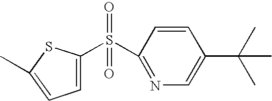 | 4-{1-[5-(5-Trifluoromethyl-pyridine- 2-sulfonyl)-thiophene-2-sulfonyl]- 2,3-dihydro-1H-indol-5-yloxy}- pyridine-2-carboxylic acid methylamide | 625 @2.43 min |
| 98 | 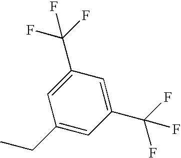 | 4-[1-(Bis-trifluoromethyl- phenylmethanesulfonyl)-2,3-dihydro- 1H-indol-5-yloxy]-pyridine-2- carboxylic acid methylamide | 560 @2.46 min |
| 99 | 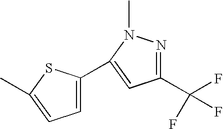 | 4-{1-[5-(Methyl-trifluoromethyl-2H- pyrazol-3-yl)-thiophene-2-sulfonyl]- 2,3-dihydro-1H-coxy}-pyridine-2- carboxylic acid methylamide | 464 @2.56 min |

### Example 100

5-[Methyl-(2-methylcarbamoyl-pyridin-4-yl)-methyl-amino]-2,3-dihydro-indole-1-carboxylic acid (3′-trifluoromethyl-phenyl)-amide

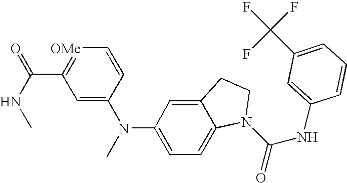

a) 4-(1-Acetyl-2,3-dihydro-1H-indol-5-ylamino)-pyridine-2-carboxylic acid methylamide. A mixture of 4 chloro-pyridine-2-carboxylic acid methyl amide (Example 1(a) 340 mg, 2 mmol) and 1-acetyl-2,3-dihydro-5-aminoindole (350 mg, 2 mmol) in isopropanol (5 ml) with ethereal HCl added (0.5 mL) was heated for 5 days at 85° in a sealed tube. LC/ms showed complete reaction. The reaction mixture was diluted with EtOAc then treated with solid NaHCO3 and filtered. The filtrate was concentrated to give a green-brown solid which was triturated with ether to afford the title compound. (699 mg, 100%) LC-MS (m/e)=311 [M+H]⁺.

b) 4-[(1-Acetyl-2,3-dihydro-1H-indol-5-yl)-methyl-amino]-pyridine-2-carboxylic acid methylamide. 4-(1-Acetyl-2,3-dihydro-1H-indol-5-ylamino)-pyridine-2-carboxylic acid methylamide (93 mg, 0.3 mmol) and CsCO₃ (600 mg) were stirred for 10 min in DMF at rt then MeI was injected (28 uL, 0.45 mmol). After 1 hr. tlc showed starting material present so an additional 10 uL of MeI was added. The reaction was stirred at rt for 18 h then diluted with water and extracted (×3) with EtOAc. The organic extracts were washed with water (×2) and brine. After drying (MgSO₄) the solvent was removed under reduced pressure to afford a yellow gum which was triturated with hot Et₂O, cooled and diluted with hexanes to afford a white solid (59 mg, 61%). LC-MS (m/e)=325[M+H]⁺@1.3 min.

c) 4-[(2,3-dihydro-1H-indol-5-yl)-methyl-amino]-pyridine-2-carboxylic acid methylamide. The 4-[(1-Acetyl-2,3-dihydro-1H-indol-5-ylmethyl-amino]-pyridine-2-carboxylic acid methylamide (59 mg, 0.182 mmol) in 2N HCl (3 ml) was stirred at 85° for 3 h. After cooling to rt the acidic solution was treated with 1 N NaOH until pH=14. The white solid which had formed was extracted into EtOAc and dried (MgSO₄). Evaporation of the solvent afforded the desired product (49.7 mg, 97%) which was used in the next reaction without further purification.

d) 5-[Methyl-(2-methylcarbamoyl-pyridin-4yl)-methyl-amino]-2,3-dihydro-indole-1-carboxylic-acid (3′-trifluoromethyl-phenyl)-amide. To a solution of 2-fluoro-5-trifluoromethyl-phenylisocyanate (40.0 mg, 0.212 mmol) in dichloromethane (2.0 mL) was added methyl-4-(2,3-dihydro-1-H-indol-5-methyl-amino)-pyridine-2-carboxylic acid methylamide (50 mg, 0.177 mmol) at rt. The reaction was stirred overnight at rt. Hplc confirmed complete reaction. The crude reaction mixture was chromatographed on reverse phase column eluting 10%-70% acetonitrile/0.1% TFA water affording after concentration and evaporation a bright yellow solid.(33 mg, 40%) LC-MS (m/e)=470.2 [M+H]⁺.

### Example 101

### 6-(2-Methylcarbamoyl-pyridin-4-yloxy)-2,3,4-trihydro-quinoline-1-carboxylic acid (3′-trifluoromethyl-phenyl)-amide

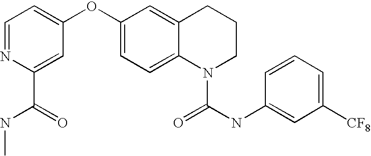

a) 4-(1,2,3,4-Tetrahydroquinoline-6-yloxy)-pyridine-2-carboxylic acid methylamide. 6-Hydroxy-1,2,3,4-tetrahydroquinoline (149 mg, 1.0 mmol) which may be prepared by the method as described by Hoenel et al in J.Chem.Soc.Perkin Tralls.I, 1933-1939 (1980), was dissolved in DMF (2 ml) and treated at rt for 5 min with potassium t-butoxide (160 mg, 1.43 eq) followed by addition of 4-Chloro-pyridine-2-carboxylic acid methylamide (171 mg, 1.0 mmol). This mixture was under microwave irradiation for 20 min at 160° C. in a Personal Chemistry synthesizer. The reaction mixture was partitioned between ethyl acetate and water and the aqueous extracted with EtOAc (×4). The combined extracts were washed with water (×3) and brine, then dried (Na₂SO₄) and evaporated under reduced pressure. The residue was purified by flash column chromatography on the silica gel (hexane/EtOAC/CH₂Cl₂ 1:2:1) to afford the title compound as an off-white solid. (239 mg, 84%) LC-MS (m/e)=284 [M+H]⁺.

b) 6-(2-Methylcarbamoyl-pyridin-4-yloxy)-2,3,4-trihydro-quinoline-1-carboxylic acid (3′-trifluoromethyl-phenyl)-amide. A solution of 3-trifluoromethyl-phenylisocyanate (61 μL, 0.40 mmol) and 4-(1,2,3,4-tetrahydroquinoline-6-yloxy)-pyridine-2-carboxylic acid methylamide (57 mg, 0.20 mmol) in DMF was stirred at rt overnight, and concentrated under the reduced pressure. The residue was purified by flash column chromatography on the silica gel (hexane/EtOAC 1:1) to afford the title compound as a white solid. (60 mg, 64%) LC-MS (m/e)=471 [M+H]⁺. ¹H NMR (400 MHz, CDCl₃) δ 8.46 (d, J=5.6 Hz, 1H), 8.2 (br s, 1H), 7.77 (m, 2H), 7.63 (d, J=9.0 Hz, 1H), 7.46-7.42 (m, 2H), 7.33 (d, J=7.6 Hz, 1H), 7.12 (m, 2H), 7.02-7.00 (m, 2), 3.89 (t, J=6.3 Hz, 2H), 3.05 (d, J=5.1 Hz, 3H), 2.83 (t, J=6.6 Hz, 2H), 2.05 (m, 2H).

### Example 102

### 6-(2-Methylcarbamoyl-pyridin-4-yloxy)-2,3,4-trihydro-quinoline-1-carboxylic acid (3′-trifluoromethyl-4′-chloro-phenyl)-amide

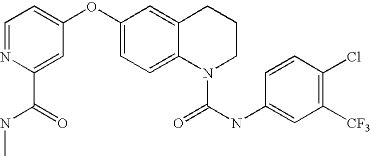

Following the procedure of Example 53(b), except substituting 4-chloro-3-trifluoromethyl-phenylisocyanate for 3-trifluoromethyl-phenylisocyanate, the title compound was prepared as a white solid (30 mg, 70%). LC-MS (m/e)=505/507 [M+H]⁺. ¹H NMR (400 MHz, CDCl₃) δ 8.43 (d, J=5.6 Hz, 1H), 8.06 (m, 1H), 7.82 (d, J=2.5 Hz, 1H), 7.70 (s, 1H), 7.66-7.63 (m, 1H), 7.44-7.34 (m, 3H), 7.11-7.08 (m, 1H), 7.03-7.00 (m, 3), 3.87 (t, J=6.3 Hz, 2H), 3.02 (d, J=5.1 Hz, 3H), 2.85 (t, J=6.7 Hz, 2H), 2.03 (m, 2H).

### Example 103

### 6-(2-Methylcarbamoyl-pyridin-4-yloxy)-2,3,4-trihydro-quinoline-1-carboxylic acid (3′,4′-difluoro-phenyl)-amide

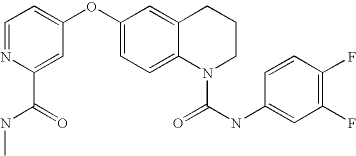

Following the procedure of Example 53(b), except substituting (3,4-difluoro-phenylisocyanate for 3-trifluoromethyl-phenylisocyanate, the title compound was prepared as a white solid (46 mg, 81%). LC-MS (m/e)=439 [M+H]⁺. 1H NMR (400 MHz; CDCl₃) δ 8.35 (d, J=5.6 Hz, 1H), 7.98 (m, 1H), 7.61 (d, J=2.5 Hz, 1H), 7.47 (ddd, J=12.4, 7.1, 2.5 Hz, 1H), 7.29 (d, J=8.4 Hz, 1H), 7.09 (s, 1H), 7.02-6.87 (m, 5H), 3.76 (t, J=6.3 Hz, 2H), 2.94 (d, J=5.1 Hz, 3H), 2.68 (t, J=6.7 Hz, 2H), 1.92 (m, 2H).

Biological Data

Compounds are tested for TIE-2 kinase and VEGFR kinase inhibition activity according to one or more of the following methods.

TIE-2 Enzyme Assay (TIE2-E)

The TIE-2 enzyme assay uses the LANCE method (Wallac) and GST-TIE2, baculovirus expressed recombinant constructs of the intracellular domains of human TIE2 (amino acids 762-1104, GenBank Accession #L06139) tagged by GST). The method measures the ability of the purified enzymes to catalyse the transfer of the γ-phosphate from ATP onto tyrosine residues in a biotinylated synthetic peptide, D1-15 (biotin-C₆-LEARLVAYEGWVAGKKKamide). This peptide phosphorylation is detected using the following procedure: for enzyme preactivation, GST-TIE2 is incubated for 30 mins at room temperature with 2 mM ATP, 5 mM MgCl2 and 12.5 mM DTT in 22.5 MM HEPES buffer (pH7.4). Preactivated GST-TIE2 is incubated for 30mins at room temperature in 96 well plates with 1 uM D1-15 peptide, 80 uM ATP, 10 mM MgCl₂, 0.1 mg/ml BSA and the test compound (diluted from a 10 mM stock in DMSO, final DMSO concentration is 2.4%) in 1 mM HEPES (pH7.4). The reaction is stopped by the addition of EDTA (final concentration 45 mM). Streptavidin linked-APC (allophycocyanin, Molecular Probe) and Europium-labeled anti-phosphorylated tyrosine antibody (Wallac) are then added at the final concentration of 17 ug/well and 2.1 ug/well, respectively. The APC signal is measured using an ARVO multilabel counter. (Wallac Berthold Japan). The percent inhibition of activity is calculated relative to blank control wells. The concentration of test compound that inhibits 50% of activity (IC₅₀) is interpolated using nonlinear regression (Levernberg-Marquardt) and the equation, y=Vmax (1−x/(K+x))+Y2, where “K” is equal to the IC₅₀. The IC₅₀ values are converted to pIC₅₀ values, i.e., -log IC₅₀ in Molar concentration.

TIE-2 Enzyme Assay (TIE2-E2

The TIE-2 enzyme assay uses the LANCE method (Wallac) and GST-TIE2, baculovirus-expressed recombinant constructs of the intracellular domains of human TIE2 (amino acids 762-1104, GenBank Accession #L06139) tagged by GST). The method measures the ability of the purified enzymes to catalyse the transfer of the γ-phosphate from ATP onto tyrosine residues in a biotinylated synthetic peptide, D1-15 (biotin-C₆-LEARLVAYEGWVAGKKKamide). This peptide phosphorylation is detected using the following procedure: for enzyme preactivation, GST-TIE2 is incubated for 2 hours at room temperature with 80 μM ATP, 10 mM MgCl₂, 0.1 mg/ml BSA, 0.01% Tween 20 and 1 mM DTT in 100 mM HEPES buffer (pH7.4). 5 nM preactivated GST-TIE2 is incubated for 2 hours at room temperature in 96 well plates with 1 uM D1-15 peptide, 80 uM ATP, 10 mM MgCl12, 0.1 mg/ml BSA, 0.01% Tween 20 and titrated test compound (diluted from a 10 mM stock in DMSO, final DMSO concentration is 2.4%) in 100 mM HEPES (pH7.4). The reaction is stopped by the addition of EDTA (final concentration 45 mM). Streptavidin linked-APC (allophycocyanin, PerkinElmer) and europium-labeled anti-phosphotyrosine antibody (PerkinElmer) are then added at the final concentration of 8 nM and 1 nM, respectively. The APC signal is measured using an Wallac Multilabel 1420 counter. (Wallac Berthold Japan). The percent inhibition of activity is calculated relative to blank control wells. The concentration of test compound that inhibits 50% of activity (IC₅₀) is interpolated using nonlinear regression (Levernberg-Marquardt) and the equation, y=Vmax (1−x/(K+x))+Y2, where “K” is equal to the IC₅₀. The IC₅₀ values are converted to pIC₅₀ values, i.e., -log IC₅₀ in Molar concentration.

TIE-2 Autophosphorylation assay (TIE2-C) The TIE-2 autophosphorylation assay uses an ELISA method and a TIE2 intracellular domain/c-fms extracellular domain (TIE2/c-fms) chimeric protein expressing mouse 3T3 cell line. This assay measures the autophosphorylation level of TIE2 protein expressed in cells. The cells are cultured in 96 well plates and grown in high glucose DMEM containing 10% serum at 37° C. in a humidified 10% CO2, 90% air incubator. On the day of the assay, the serum containing medium is removed from the cells and replaced with serum free medium for one hour. The test compound (diluted from a 10 mM stock in DMSO, fmal DMSO concentration is 0.1%) is incubated with TIE2/c-fms expressing cells for 30 minutes in serum free DMEM. Intrinsic cellular dephosphorylation of the receptor is blocked by the addition of the tyrosine phosphatase inhibitor, sodium orthovanadate, from a 100 mM aqueous stock to a final concentration of 1 mM. The culture media is removed by aspiration and the cells incubated for 30 to 60 mins on ice with lysis buffer containing 137 mM NaCl, 2 mM EDTA, 10% glycerol, 1 mM sodium ortho vanadate, 1× tyrosine phosphatase inhibitor cocktail (Sigma) and complete protease inhibitor cocktail (Roche) in 20 mM Tris-HCl (pH8.0). The cell extracts are transferred into Rat anti-c-fms antibody (Zymed—clone 12-2d6)(2.5 mg/ml) coated 96 well plates and incubated for 12 hrs at 4 degrees. The extracts are removed by aspiration and the plate, washed in a buffer comprising PBS, 0.05% Tween-20, 0.05% NP-40 and 5% SuperBlock (Pierce) followed by incubation with an HRP (horseradish peroxidase) conjugated anti-phosphotyrosine antibody, (Upstate Biotech) The plates are again washed with the aforementioned wash buffer and the colorimetric HRP substrate, TMB is added. The reaction progresses for 90 seconds and is stopped with the addition of 2M H₂SO₄. The optical density at 450 nm derived from HRP catalyzed TMB is measured with a plate reader capable of reading at the appropriate wavelength (e.g. SpectroMax from Molecular Dynamics). The percent inhibition of activity is calculated relative to non-vanadate treated control wells. The concentration of test compound that inhibits 50% of activity (IC₅₀) is interpolated using nonlinear regression (Levernberg-Marquardt) and the equation, y=Vmax (1−x/(K+x))+Y2, where “K” is equal to the IC₅₀.

Tie2 Fluorescence Polarization Kinase Activity Assay: (TIE2-FP)

Activation of recombinant Tie2 activation: Recombinant GST-Tie2 is activated by incubating the enzyme in 20 mM Tris-HCl, pH 7.5, 12 mM MgCl₂, 100 mM NaCl, 20 μM sodium vanidate, 1 mM DTT and 300 μM ATP at room temperature for 2 hours. The activation mixture is then passed through a NAP-25 desalting column (Pharmacia Biotech cat. no. 17-0852-02) to remove the free ATP. The activated enzyme is stored as aliquots at −80° C. in 20 mM Tris-HCl, pH 7.5 and 100 mM NaCl.

Assay conditions: The final assay conditions are 50 mM HEPES, pH 7.5, 5% DMSO (when screening compounds), 200 μM ATP, 5 mM MgCl₂, 1 M DTT, 50 μM sodium vanidate, 1 nM activated enzyme, and 200 μM peptide. IC₅₀'s of compounds are measured under subsaturating ATP (200 μM) and varying concentrations of activated Tie2 and peptide substrate (RFWKYEFWR-OH; MW 1873 Da, TFA salt). Panvera Anti-phosphotyrosine antibody (Cat#P2840) and PTK Green Tracer (Cat#P2842) are used to detect the phosphorylated peptide. Polarization is measured on a TECAN Polarion in 138-second cycles for 30 minutes at room temperature. IC₅₀'s are then determined from the % polarization using normal calculation methods. The IC₅₀ values are converted to pIC₅₀ values, i.e., -log IC₅₀ in Molar concentration.

VEGF-R2 enzyme assay (VEGF-E): The VEGF enzyme assay uses the LANCE method (Wallac) and GST-VEGFR2, baculovirus expressed recombinant constructs of the intracellular domains of human TIE2 tagged by GST. The method measures the ability of the purified enzymes to catalyse the transfer of the γ-phosphate from ATP onto tyrosine residues in a biotinylated synthetic peptide, (biotin-aminohexyl-EEEEYFELVAKKKK-NH2). This peptide phosphorylation is detected using the following procedure: GST-VEGFR2 is incubated for 40-60 mins at room temperature with 75 uM ATP, 5 mM MgCl2, 0.1 mM DTT, 0.1 mg/mL BSA and the test compound (diluted from a 10 mM stock in DMSO for desired concentration) in 100 mM HEPES buffer. The reaction is stopped by the addition of EDTA (final concentration 50 mM). Streptavidin linked-APC (allophycocyanin, Molecular Probe) and Europium-labeled anti-phosphorylated tyrosine antibody (Wallac) are then added at the fmal concentration of 15 nM and 1 nM, respectively. The APC signal is measured using an ARVO multilabel counter (Wallac Berthold, Japan). The percent inhibition of activity is calculated relative to blank control wells. The concentration of test compound that inhibits 50% of activity (IC₅₀) is interpolated using nonlinear regression (Levernberg-Marquardt) and the equation, y=Vₘₐₓ(1−x/(K+x))+Y2, where “K” is equal to the IC₅₀. The IC₅₀ values are converted to pIC₅₀ values, i.e., -log IC₅₀ in Molar concentration.

VEGF-R2 enzyme assay (VEGF-E2): The VEGF enzyme assay uses the LANCE method (Wallac) and GST-VEGFR2, baculovirus expressed recombinant constructs of the intracellular domains of human TIE2 tagged by GST. The method measures the ability of the purified enzymes to catalyse the transfer of the γ-phosphate from ATP onto tyrosine residues in a biotinylated synthetic peptide, (biotin-aminohexyl-EEEEYFELVAKKKK-NH2). This peptide phosphorylation is detected using the following procedure: GST-VEGFR2 is incubated for 40-60 mins at room temperature with 75 uM ATP, 5 mM MgCl₂, 0.1 mM DTT, 0.1 mg/mL BSA and the test compound (diluted from a 10 mM stock in DMSO for desired concentration) in 100 mM HEPES buffer. The reaction is stopped by the addition of EDTA (final concentration 50 mM). Streptavidin linked-APC (allophycocyanin, Molecular Probe) and Europium-labeled anti-phosphorylated tyrosine antibody (Wallac) are then added at the final concentration of 15 nM and 1 nM, respectively. The APC signal is measured using an ARVO multilabel counter (Wallac Berthold, Japan). The percent inhibition of activity is calculated relative to blank control wells. The concentration of test compound that inhibits 50% of activity (IC₅₀) is interpolated using nonlinear regression (Levernberg-Marquardt) and the equation, y=Vₘₐₓ(1−x/(K+x))+Y2, where “K” is equal to the IC₅₀. The IC₅₀ values are converted to pIC₅₀ values, i.e., -log IC₅₀ in Molar concentration.

VEGF-friven cellular proliferation assay: BrdU incorporation assay (VEGF-C)

Human umbilical cord endothelial cells (HUVEC, Clonetics, CC2519) are passaged in Type I collagen-coated 100-mm petri dishes in EGM-MV medium (Clonetics, CC3125) at 37 C in a humidified 5% CO2, 95% air incubator. (HUVEC passaged more than 6 times in vitro are discarded and not subjected to assaying.) The cells are harvested using trypsin/EDTA, counted using a haemocytometer and plated at 5000 cells/well in a Type I-collagen coated 96-well plate (Becton Dickinson, 354407) in M199 medium (Gibco BRL, 12340-030) containing 5% FBS (Hyclone, A 1115-L) and gentamicin (at 50 ug/ml, Gibco BRL). After incubation overnight at 37° C., the media are replaced with 100 ul of M199 serum-free medium containing compounds at various concentrations with 0.6% DMSO and gentamicin. The compounds are diluted in serum-free M199 medium from 10 mM stock solutions prepared in 100% DMSO. After a 30 min incubation at 37° C., the cells are fed with 100 ul of serum-free M199 medium containing gentamicin, 0.2% culture-grade bovine serum albumin (BSA, Sigma A1993) and 20 mg/ml of VEGF (R&D systems, 293-VE) or 0.6 mg/ml of basic FGF (R&D systems, 233-FB), and cultured at 37° C. for another 24 h. The cells are pulsed with bromodeoxyuridine (BrdU at 10 uM in serum-free M199) at 37° C. for an additional 24 h. The incorporation of BrdU into the proliferating HUVEC are analyzed using BrdU Cell Proliferation ELISA (Roche Molecular Biochemicals, 1647229) according to the manufacturer's protocols. The optical density at 450 nm is measured with a multilabel counter (ARVO SX, Wallac). The percent inhibition of cell growth is calculated relative to blank control wells. The concentration of test compound that inhibits 50% of cell growth (IC₅₀) is interpolated using nonlinear regression (Levernberg-Marquardt) and the equation, y=Vmax (1−x/(K+x))+Y2, where “K” is equal to the IC₅₀. The IC₅₀ values are converted to pIC₅₀ values, i.e., -log IC₅₀ in Molar concentration.

VEGFR-3—Homogenous Time-Resolved Fluorescence Assay (VEGFR-3-HTRF)

This assay assesses Vascular Endothelial Growth Factor 3 (VEGFR3) tyrosine kinase inhibitory activity in substrate phosphorylation assays. The assay examines the ability of small molecule organic compounds to inhibit the tyrosine phosphorylation of a peptide substrate.

The substrate phosphorylation assays use the VEGFR3 catalytic domain, which is expressed in Sf. 9 insect cells as an amino-terminal GST-tagged fusion protein. The catalytic domain of human VEGFR3 (AA residues #819-1298 based upon GenBank Accession #XM003852) is cloned by PCR from human Placenta Marathon Ready cDNA (Clontech). The PCR product is subcloned into pFastBacl vector containing an N-terminal GST tag. The resulting pFB/GST/VEGFR3icd vector is used to generate a recombinant baculovirus for protein expression. The VEGFR3 catalytic domain translated sequence is:

MSPILGYWKI KGLVQPTRLL LEYLEEKYEE HLYERDEGDK
WRNKKFELGL EFPNLPYYID GDVKLTQSMA IIRYIADKHN
MLGGCPKERA EISMLEGAVL DIRYGVSRIA YSKDFETLKV
DFLSKLPEML KMFEDRLCHK TYLNGDHVTH PDFMLYDALD
VVLYMDPMCL DAFPKLVCFK KRIEATPQID KYLKSSKYIA
WPLQGWQATF GGGDHPPKSD LLVPRGSPEF KGLPGEVPLE
EQCEYLSYDA SQWEFPRERL HLGRVLGYGA FGKVVEASAF
GIHKGSSCDT VAVKMLKEGA TASEQRALMS ELKILIHIGN
HLNVVNLLGA CTKPQGPLMV IVEFCKYGNL SNFLRAKRDA
FSPCAEKSPE QRGRFRAMVE LARLDRRRPG SSDRVLFARF
SKTEGGARRA SPDQEAEDLW LSPLTMEDLV CYSFQVARGM
EFLASRKCIH RDLAARNILL SESDVVKICD FGLARDIYKD
PDYVRKGSAR LPLKWMAPES IFDKVYTTQS DVWSFGVLLW
EIFSLGASPY PGVQINEEFC QRLRDGTRMR APELATPAIR
RIMLNCWSGD PKARPAFSEL VEILGDLLQG RGLQEEEEVC
MAPRSSQSSE EGSFSQVSTM ALHIAQADAE DSPPSLQRHS
LAARYYNWVS FPGCLARGAE TRGSSRMKTF EEFPMTPTTY
KGSVDNQTDS GMVLASEEFE QIESRHRQES GFR

Autophosphorylation allows enzymes to be fully activated prior to addition to peptide substrates. The assays are performed using enzyme that has been activated by autophosphorylation via preincubation in buffer with ATP and magnesium. Activated enzyme is then diluted and added to titrated compound and the substrate mix.

200 nM VEGFR3 enzyme is activated for 45 minutes at room temperature by incubating the enzyme in buffer containing 100 mM HEPES (pH7.2), 75 μM ATP, 0.3 mM DTT, 0.1 mg/mL BSA, and 10 mM MgCl₂. After activation, VEGFR3 is diluted 100-fold into 2× dilution buffer: 200 mM HEPES (pH 7.5), 0.2 mg/mL BSA, 0.6 mM DTT. 20 μL of the diluted enzyme mix is added to 20 μL of 2× substrate mix (150 μM ATP, 20 mM MgCl₂ 0.72 μM biotinylated peptide) in the assay plates. Final assay conditions are: 100 mM HEPES (pH 7.2), 75 μM ATP, 20 mM MgCl₂, 0.1 mg/mL BSA, 0.3 mM DTT, 0.36 μM biotinylated peptide, and 1 nM VEGFR3 enzyme. Assay plates are incubated for 1.5 hours at room temperature before the addition of 30 μL 100 mM EDTA to the wells to stop the enzymatic reaction. 40 μL/well of HTRF mix are then added to the assay plates for the detection of phosphorylated substrate. Final assay concentrations are: 100 mM HEPES (pH7.2), 0.1 mg/mL BSA, 15 nM streptavidin-labeled allophycocyanin (PerkinElmer), and 1 nM europium-labeled anti-phosphotyrosine antibody (PerkinElmer). Assay plates are left unsealed and are counted in a Wallac Multilabel Counter 1420 (PerkinElmer).

The data for dose responses are plotted as % Control calculated with the data reduction formula (100)(U1-C2)/(C1-C2) versus concentration of compound where U is the unknown value, C1 is the average control value obtained for DMSO, and C2 is the average control value obtained for 0.1 M EDTA. Data are fitted to the curve described by: y=((Vmax)(x)/(K+x)) where Vmax is the upper asymptote and K is the IC50.

The compounds of Examples 3, 4, 6 and 7 demonstrated inhibition of TIE2 kinase with an IC₅₀ of less than 250 nm. The compounds of Examples 1-8 demonstrated inhibition of VEGFR2 kinase with an IC₅₀ of less than 250 nm. The compound of Example 8 demonstrated inhibition of Raf kinase with an IC₅₀ of less than 250 nm.

We claim:

1. A compound of Formula I: 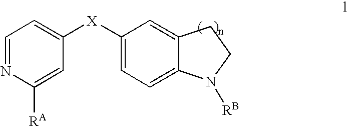
wherein:
n is an integer of 1, 2, or 3;
R^{A} is —CONHR¹, —NHR¹, —NHCOR¹, —NHCONHR¹, —NHCO₂R¹, —NHSO₂R¹ or —NHSO₂NHR¹;
wherein R¹ is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl- or heteroaryl-C₁-C₄ alkyl- group,
where said optionally substituted R¹ group is optionally substituted with one or more substituents independently selected from halogen, —R^{1a}, —OR^{1a}, —SR^{1a}, —SO₂R^{1c}—NR^{1a}R^{1b}, cyano, nitro, —COR^{1c}, —CO₂R^{1a}, —NR^{1b}COR^{1a}, —CONR^{1a}R^{1b}, —NR^{1b}SO₂R^{1c}, and —SO₂NR^{1a}R^{1b},
where R^{1a} is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,
R^{1b} is hydrogen or unsubstituted C₁-C₄ alkyl, and
R^{1c} is an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,
where each optionally substituted R^{1a} group and R^{1c} group is independently optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), -C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,
or R^{1a} and R^{1b}, together with the nitrogen atom to which they are attached, form an optionally substituted heterocycyl or heteroaryl ring which optionally contains one or more additional heteroatom moieties selected from O, S, SO, SO₂, N and N→O, wherein said optionally substituted heterocycyl or heteroaryl ring is optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,
X is NR², O, S, SO or SO₂,
wherein R is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl- or heteroaryl-C₁-C₄ alkyl- group,
where said optionally substituted R² group is optionally substituted with one or more substituents independently selected from halogen, —R^{2a}, —OR^{2a}, —SR^{2a}, —SO₂R^{2c}—NR^{2a}R^{2b}, cyano, nitro, —COR^{2c}, —CO₂R^{2a}, —NR^{2b}COR^{2a}, —CONR^{2a}R^{2b}, —NR^{2b}SO₂R^{2c}, and —SO₂NR^{2a}R^{2b},
where R²a is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,
R^{2b} is hydrogen or unsubstituted C₁-C₄ alkyl, and
R^{2c} is an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,
where each optionally substituted R^{2a} group and R^{2c} group is independently optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,
or R^{2a} and R^{2b}, together with the nitrogen atom to which they are attached, form an optionally substituted heterocycyl or heteroaryl ring which optionally contains one or more additional heteroatom moieties selected from O, S, SO, SO₂, N and N→O, wherein said optionally substituted heterocycyl or heteroaryl ring is optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,
R^{B} is —CONHR³, —SO₂R³; —CO₂R³, —COC(R⁴R⁵)R³,
wherein R³ is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl- or heteroaryl-C₁-C₄ alkyl- group,
where said optionally substituted R³ group is optionally substituted with one or more substituents independently selected from halogen, —R^{3a}, —OR^{3a}, —SR^{3a}, —SO₂R^{3c}—NR^{3a}R^{3b}, cyano, nitro, —COR^{3c}, —CO₂R^{3a}, —NR^{3b}COR^{3a}, —CONR^{3a}R^{3b}, —NR^{3b}SO₂R^{3c}, and —SO₂NR^{3a}R^{3b},
where R^{3a} is hydrogen or an optionally substituted C₁-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,
R^{3b} is hydrogen or unsubstituted C₁-C₄ alkyl, and
R^{3c} is an optionally substituted C,-C₆ alkyl, C₂-C₆ alkenyl, C₂-C₆ alkynyl, aryl, C₃-C₇ cycloalkyl, heteroaryl, heterocyclyl, aryl-C₁-C₄ alkyl-, C₃-C₇ cycloalkyl-C₁-C₄ alkyl-, heteroaryl-C₁-C₄ alkyl-, heterocycyl-C₁-C₄ alkyl-, aryl-C₂-C₄ alkenyl-, C₃-C₇ cycloalkyl-C₂-C₄ alkenyl-, heteroaryl-C₂-C₄ alkenyl-, heterocycyl-C₂-C₄ alkenyl-, aryl-C₂-C₄ alkynyl-, C₃-C₇ cycloalkyl-C₂-C₄ alkynyl-, heteroaryl-C₂-C₄ alkynyl-, or heterocycyl-C₂-C₄ alkynyl- group,
where each optionally substituted R^{3a} group and R^{3c} group is independently optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C,-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄ alkyl is unsubstituted C₁-C₄ alkyl,
or R^{3a} and R^{3b}, together with the nitrogen atom to which they are attached, form an optionally substituted heterocycyl or heteroaryl ring which optionally contains one or more additional heteroatom moieties selected from O, S, SO, SO₂, N and N→O, wherein said optionally substituted heterocycyl or heteroaryl ring is optionally substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, SO₂NH(C₁-C₄ alkyl), —NHS(O)₂(C₁-C₄ alkyl), and —NHS(O)₂(C₁-C₄ haloalkyl), where said C₁-C₄alkyl is unsubstituted C₁-C₄alkyl, and
R⁴ and R⁵ are independently selected from hydrogen and unsubstituted C₁-C₄ alkyl,
or R⁴ and R⁵, taken together with the carbon atom to which they are attached, represent an optionally substituted 3-6-membered saturated carbocyclic ring, where said optionally substituted 3-6-membered ring is substituted with one or more substituents independently selected from C₁-C₄ alkyl, C₁-C₄ haloalkyl, —OC₁-C₄ alkyl, —OC₁-C₄ haloalkyl, halogen, —OH, —NH₂, —N(C₁-C₄ alkyl)(C₁-C₄ alkyl), —NH(C₁-C₄ alkyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —C(O)C₁-C₄ alkyl, —C(O)C₁-C₄ haloalkyl, —OC(O)C₁-C₄ alkyl, —OC(O)C₁-C₄ haloalkyl, —SO₂(C₁-C₄ alkyl), —SO₂(C₁-C₄ haloalkyl), —SO₂NH₂, —SO₂NH(C₁-C₄ alkyl), —NHS(O)₂(C₁-C₄ alkyl), and —NHS(O)₂(C₁-C₄ haloalkyl), where said C₁-C₄alkyl is unsubstituted C₁-C₄alkyl.,
or a salt, solvate, or physiologically functional derivative thereof.

2. A compound according to claim 1, wherein n is 1 or 2.

3. A compound according to claim 1, wherein X is O or NR2.

4. A compound according to claim 1, having the formula:

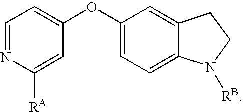

5. A compound according to claim 1, having the formula:

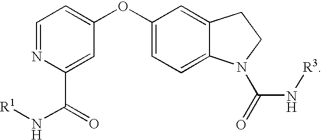

6. A compound according to claim 1, wherein R^{A} is —CONHR¹, —NHCOR¹, or —NHSO₂R¹, where R¹ is C₁-C₆ alkyl, aryl, heteroaryl, heterocycyl, aryl-C₁-C₄ alkyl-, heteroaryl-C₁-C₄ alkyl-, or heterocycyl-C₁-C₄ alkyl-, wherein said C₁-C₆ alkyl is optionally substituted with one ore more substituents independently selected from —NH₂, —N(C₁-C₄ alkyl)(C₁-C₄ alkyl), and —NH(C₁-C₄ alkyl), or said aryl, heteroaryl or heterocycyl or the aryl, heteroaryl or heterocycyl moiety of said aryl-C₁-C₄ alkyl-, heteroaryl-C₁-C₄ alkyl-, or heterocycyl-C₁-C₄ alkyl- is unsubstituted or substituted by one or more substituents independently selected from C₁-C₄ alkyl, C₁-C₄ haloalkyl and halogen.

7. A compound according to claim 1, wherein R^{B} is —CONHR³ or —SO₂R³; where R³ is aryl or heteroaryl, wherein said aryl or heteroaryl is unsubstituted or substituted by one or more substituents independently selected from C₁-C₄ alkyl, C₁-C₄ haloalkyl, halogen, C₁-C₆ alkyl, C₃-C₆ cycloalkyl, aryl, heteroaryl and heterocycyl.

8. A compound according to claim 1, wherein R^{A} is —CONHR¹, where R¹ is C₁-C₆ alkyl, aryl, heteroaryl, heterocycyl, aryl-C₁-C₄ alkyl-, heteroaryl-C₁-C₄ alkyl-, or heterocycyl-C₁-C₄ alkyl-, wherein said C₁-C₆ alkyl is optionally substituted with one ore more substituents independently selected from —NH₂, —N(C₁-C₄ alkyl)(C₁-C₄ alkyl), and —NH(C₁-C₄ alkyl), or said aryl, heteroaryl or heterocycyl or the aryl, heteroaryl or heterocycyl moiety of said aryl-C₁-C₄ alkyl-, heteroaryl-C₁-C₄ alkyl-, or heterocycyl-C₁-C₄ alkyl- is unsubstituted or substituted by one or more substituents independently selected from C₁-C₄ alkyl, C₁-C₄ haloalkyl and halogen.

9. A compound according to claim 1, wherein R^{B} is —CONHR , where R³ is aryl or heteroaryl, wherein said aryl or heteroaryl is unsubstituted or substituted by one or more substituents independently selected from C₁-C₄ alkyl, C₁-C₄ haloalkyl, halogen, C₁-C₆ alkyl, C₃-C₆ cycloalkyl, aryl, heteroaryl and heterocycyl.

10. A compound according to claim 1, wherein R^{A} is —CONHR¹, —NHCOR¹, —NHSO₂R¹, where R¹ is C₁-C₆ alkyl, aryl, heteroaryl, heterocycyl, aryl-C₁-C₄ alkyl-, heteroaryl-C₁-C₄ alkyl-, or heterocycyl-C₁-C₄ alkyl-, wherein said C₁-C₆ alkyl is optionally substituted with one or more substituents independently selected from —NH₂, —N(C₁-C₄ alkyl)(C₁-C₄ alkyl), —NH(C₁-C₄ alkyl), or said aryl, heteroaryl or heterocycyl or the aryl, heteroaryl or heterocycyl moiety of said aryl-C₁-C₄ alkyl-, heteroaryl-C₁-C₄ alkyl-, or heterocycyl-C₁-C₄ alkyl- is unsubstituted or substituted by one or more substituents independently selected from C₁-C₄ alkyl, C₁-C₄ haloalkyl and halogen., and R^{B} is —CONHR³ or —SO₂NHR³; where R³ is aryl or heteroaryl, wherein said aryl or heteroaryl is unsubstituted or substituted by one or more substituents independently selected from C₁-C₄ alkyl, C₁-C₄ haloalkyl, halogen, C₁-C₆ alkyl, C₃-C₆ cycloalkyl, aryl, heteroaryl and heterocycyl;
or a salt, solvate, or physiologically functional derivative thereof.

11. A compound according to claim 10, wherein
R¹ is methyl, ethyl, phenyl, benzyl, phenethyl, N,N diethylaminopropyl, N-methyl-piperidinyl, piperidinyl-ethyl, pyrrolidinyl-butyl, morpholino-ethyl, or morpholino-propyl; and
R³ is substituted phenyl or substituted isoxazolyl, where said phenyl or isoxazolyl is substituted by one or more substituents independently selected from F, Cl, CF₃, or tert-butyl;
or a salt, solvate, or physiologically functional derivative thereof.

12. A compound according to claim 1 wherein
n is 1 or 2;
R^{A} is —CONHR¹, where R¹ is methyl, ethyl, phenyl, benzyl, phenethyl, N,N diethylaminopropyl, N-methyl-piperidinyl, piperidinyl-ethyl, pyrrolidinyl-butyl, morpholino-ethyl, or morpholino-propyl;
X is O; and
R^{B} is —CONHR³, where R³ is substituted phenyl or substituted isoxazolyl, where said phenyl or isoxazolyl is substituted by one or more substituents independently selected from F, Cl, CF₃, or tert-butyl;
or a salt, solvate, or physiologically functional derivative thereof.

13. A compound according to claim 1 wherein
n is 1;
R^{A} is —CONHR¹, where R¹ is methyl, ethyl, phenyl, benzyl, phenethyl, N,N diethylaminopropyl, N-methyl-piperidinyl, piperidinyl-ethyl, pyrrolidinyl-butyl, morpholino-ethyl, or morpholino-propyl;
X is O; and
R^{B} is —CONHR³, where R³ is substituted phenyl or substituted isoxazolyl, where said phenyl or isoxazolyl is substituted by one or more substituents independently selected from F, Cl, CF₃, or tert-butyl;
or a salt, solvate, or physiologically functional derivative thereof.

14. A pharmaceutical composition, comprising:
a) a therapeutically effective amount of a compound according to claim 1, or a salt, solvate, or a physiologically functional derivative thereof, and
b) one or more of pharmaceutically acceptable carriers, diluents and excipients,
c) the composition optionally further comprising an additional agent: selected from anti-neoplastic agents, agents which inhibit angiogenesis, or a combination thereof.

15. A method of treating a mammal having a disorder mediated by at least one of inappropriate TIE-2 kinase, VEGFR-2 kinase, VEGFR-3 kinase or Raf kinase activity comprising administering to said mammal a therapeutically effective amount of a compound according to claim 1, or a salt, solvate, or a physiologically functional derivative thereof.

16. A method of treating a mammal having a cancer, comprising administering to said mammal a therapeutically effective amount of a compound according to claim 1, or a salt, solvate, or a physiologically functional derivative thereof,
the method optionally further comprising administering a therapeutically effective amount of at least one additional anti-cancer therapy, for example, wherein the additional anti-cancer therapy is administered before, concomitantly with, or after the administration of the compound according to claim 1, salt, solvate or physiologically functional derivative thereof.

17. A method of treating a mammal having a disease which is characterized by cellular proliferation in the area of disorders associated with neo-vascularization and/or vascular permeability in a mammal, comprising administering to said mammal a therapeutically effective amount of compound according to claim 1, or a salt, solvate, or a physiologically functional derivative thereof.

18. A method of treating a mammal having a disorder mediated by at least one of inappropriate TIE-2 kinase, VEGFR-2 kinase, or VEGFR-3 kinase activity, comprising administering to said mammal therapeutically effective amounts of:
a) a compound according to claim 1, or a salt, solvate or physiologically functional derivative thereof, and
b) an agent to inhibit growth factor receptor function,
wherein the agent to inhibit growth factor receptor function is selected from an agent that inhibits the function of platelet derived growth factor receptor, the function of epidermal growth factor receptor, the function of the erbB2 receptor, the function of the erbB4 receptor, the function of a VEGF receptor, and/or the function of the TIE-2 receptor,
wherein:
i) the agent to inhibit growth factor receptor function inhibits the function of the epidermal growth factor receptor and the erbB2 receptor;
ii) the agent to inhibit growth factor receptor function inhibits the function of at least two of the epidermal growth factor receptor, the erbB2 receptor, and the erbB4 receptor; or
iii) the agent to inhibit growth factor receptor function inhibits the function of at least one of the VEGF receptor and the TIE-2 receptor.

19. A method of treating a mammal having a disorder characterized by inappropriate angiogenesis, comprising administering to said mammal a therapeutically effective amount of a compound according to claim 1, or a salt, solvate or physiologically functional derivative thereof,
wherein the inappropriate angiogenesis results from at least one of inappropriate VEGFR-2 kinase, VEGFR-3 kinase, or TIE-2 kinase activity, and
the method optionally further comprising administering a therapeutically effective amount of a VEGFR2 inhibitor.

20. The method according to claim 15, wherein the disorder is selected from cancer and diseases afflicting mammals which are characterized by cellular proliferation and being in the area of disorders associated with neo-vascularization and/or vascular permeability.


SEQUENCE LISTING (4 sequences)

SEQ ID NO: 1 - protein, 17 aa, Homo sapien
LEARLVAYEGWVAGKKK

SEQ ID NO: 2 - protein, 10 aa, Homo sapien
RFWKYEFWRH

SEQ ID NO: 3 - protein, 16 aa, Homo sapien
EEEEYFELVAKKKKNH

SEQ ID NO: 4 - protein, 713 aa, Homo sapien
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLLVPRGSPEFKGLPGEVPLEEQCEYLSYDASQWEFPRERLHLGRVLGYGAFGKVVEASAFGIHKGSSCDTVAVKMLKEGATASEQRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR